SEQUENCE LISTING (55 sequences)

SEQ ID NO: 1 - protein, 4 aa, Artificial sequence
  PBAE-modifying oligopeptide
CHHH

SEQ ID NO: 2 - protein, 5 aa, Artificial sequence
  PBAE-modifying oligopeptide
CHHHH

SEQ ID NO: 3 - protein, 6 aa, Artificial sequence
  PBAE-modifying oligopeptide
CHHHHH

SEQ ID NO: 4 - protein, 4 aa, Artificial sequence
  PBAE-modifying oligopeptide
CRRR

SEQ ID NO: 5 - protein, 5 aa, Artificial sequence
  PBAE-modifying oligopeptide
CRRRR

SEQ ID NO: 6 - protein, 6 aa, Artificial sequence
  PBAE-modifying oligopeptide
CRRRRR

SEQ ID NO: 7 - protein, 4 aa, Artificial sequence
  PBAE-modifying oligopeptide
CKKK

SEQ ID NO: 8 - protein, 5 aa, Artificial sequence
  PBAE-modifying oligopeptide
CKKKK

SEQ ID NO: 9 - protein, 6 aa, Artificial sequence
  PBAE-modifying oligopeptide
CKKKKK

SEQ ID NO: 10 - protein, 4 aa, Artificial sequence
  PBAE-modifying oligopeptide
CEEE

SEQ ID NO: 11 - protein, 5 aa, Artificial sequence
  PBAE-modifying oligopeptide
CEEEE

SEQ ID NO: 12 - protein, 6 aa, Artificial sequence
  PBAE-modifying oligopeptide
CEEEEE

SEQ ID NO: 13 - protein, 4 aa, Artificial sequence
  PBAE-modifying oligopeptide
CDDD

SEQ ID NO: 14 - protein, 5 aa, Artificial sequence
  PBAE-modifying oligopeptide
CDDDD

SEQ ID NO: 15 - protein, 6 aa, Artificial sequence
  PBAE-modifying oligopeptide
CDDDDD

SEQ ID NO: 16 - protein, 4 aa, Artificial sequence
  PBAE-modifying oligopeptide
CHRH

SEQ ID NO: 17 - protein, 4 aa, Artificial sequence
  PBAE-modifying oligopeptide
CHRR

SEQ ID NO: 18 - protein, 4 aa, Artificial sequence
  PBAE-modifying oligopeptide
CHKH

SEQ ID NO: 19 - protein, 4 aa, Artificial sequence
  PBAE-modifying oligopeptide
CHKK

SEQ ID NO: 20 - protein, 4 aa, Artificial sequence
  PBAE-modifying oligopeptide
CHEH

SEQ ID NO: 21 - protein, 4 aa, Artificial sequence
  PBAE-modifying oligopeptide
CHEE

SEQ ID NO: 22 - protein, 4 aa, Artificial sequence
  PBAE-modifying oligopeptide
CHDH

SEQ ID NO: 23 - protein, 4 aa, Artificial sequence
  PBAE-modifying oligopeptide
CHDD

SEQ ID NO: 24 - protein, 4 aa, Artificial sequence
  PBAE-modifying oligopeptide
CRHR

SEQ ID NO: 25 - protein, 4 aa, Artificial sequence
  PBAE-modifying oligopeptide
CRHH

SEQ ID NO: 26 - protein, 4 aa, Artificial sequence
  PBAE-modifying oligopeptide
CRKR

SEQ ID NO: 27 - protein, 4 aa, Artificial sequence
  PBAE-modifying oligopeptide
CRKK

SEQ ID NO: 28 - protein, 4 aa, Artificial sequence
  PBAE-modifying oligopeptide
CRER

SEQ ID NO: 29 - protein, 4 aa, Artificial sequence
  PBAE-modifying oligopeptide
CREE

SEQ ID NO: 30 - protein, 4 aa, Artificial sequence
  PBAE-modifying oligopeptide
CRDR

SEQ ID NO: 31 - protein, 4 aa, Artificial sequence
  PBAE-modifying oligopeptide
CRDD

SEQ ID NO: 32 - protein, 4 aa, Artificial sequence
  PBAE-modifying oligopeptide
CKHK

SEQ ID NO: 33 - protein, 4 aa, Artificial sequence
  PBAE-modifying oligopeptide
CKHH

SEQ ID NO: 34 - protein, 4 aa, Artificial sequence
  PBAE-modifying oligopeptide
CKRK

SEQ ID NO: 35 - protein, 4 aa, Artificial sequence
  PBAE-modifying oligopeptide
CKRR

SEQ ID NO: 36 - protein, 4 aa, Artificial sequence
  PBAE-modifying oligopeptide
CDHD

SEQ ID NO: 37 - protein, 4 aa, Artificial sequence
  PBAE-modifying oligopeptide
CDHH

SEQ ID NO: 38 - protein, 4 aa, Artificial sequence
  PBAE-modifying oligopeptide
CDRD

SEQ ID NO: 39 - protein, 4 aa, Artificial sequence
  PBAE-modifying oligopeptide
CDRR

SEQ ID NO: 40 - protein, 4 aa, Artificial sequence
  PBAE-modifying oligopeptide
CDKD

SEQ ID NO: 41 - protein, 4 aa, Artificial sequence
  PBAE-modifying oligopeptide
CDKK

SEQ ID NO: 42 - protein, 4 aa, Artificial sequence
  PBAE-modifying oligopeptide
CEHE

SEQ ID NO: 43 - protein, 4 aa, Artificial sequence
  PBAE-modifying oligopeptide
CEHH

SEQ ID NO: 44 - protein, 4 aa, Artificial sequence
  PBAE-modifying oligopeptide
CERE

SEQ ID NO: 45 - protein, 4 aa, Artificial sequence
  PBAE-modifying oligopeptide
CERR

SEQ ID NO: 46 - protein, 4 aa, Artificial sequence
  PBAE-modifying oligopeptide
CEDE

SEQ ID NO: 47 - protein, 4 aa, Artificial sequence
  PBAE-modifying oligopeptide
CEDD

SEQ ID NO: 48 - protein, 12 aa, Human immunodeficiency virus
GRKKRRQRRRPQ

SEQ ID NO: 49 - protein, 18 aa, Drosophila melanogaster
RQIKIWFQNRRMKWKKGG

SEQ ID NO: 50 - protein, 13 aa, Simian virus 40
CGYGPKKKRKVGG

SEQ ID NO: 51 - protein, 21 aa, Vespula lewisii
AGYLLGKINLKALAALAKKIL

SEQ ID NO: 52 - protein, 21 aa, Artificial sequence
  Cell penetrating peptide
KETWWETWWTEWSQPKKKRRV

SEQ ID NO: 53 - protein, 18 aa, Artificial sequence
  Multiple antigenic peptide
KLALKLALKALKAALKLA

SEQ ID NO: 54 - protein, 15 aa, Feline leukemia virus
RRRRNRTRRNRRRVR

SEQ ID NO: 55 - protein, 18 aa, Mus musculus
LLIILRRRIRKQAHAHSK

(19) United States
(12) Patent Application Publication
(10) Pub. No.: US 2022/0389158 A1
(43) Pub. Date: Dec. 8, 2022

(54) DMSO-Free Synthesis of Oligopeptide-Modified Poly(Beta-Amino Ester)s and Their Use in Nanoparticle Delivery Systems

(71) Applicant: Ixaka France, Villejuif (FR)

(72) Inventors: Özgül TEZGEL, Paris (FR); Frédéric MOURLANE, Nice (FR); Cécile BAUCHE, Paris (FR); Renaud VAILLANT, Gentilly (FR)

(21) Appl. No.: 17/762,133

(22) PCT Filed: Sep. 21, 2020
(86) PCT No.: PCT/IB2020/000783

§ 371 Date: Mar. 21, 2022

(51) Int. Cl. C08G 63/91; A61K 47/59; A61K 47/69; C08G 63/685

(52) U.S. Cl. CPC C08G 63/91; A61K 47/593; A61K 47/6925; C08G 63/685

(57) ABSTRACT

Methods for synthesizing and purifying oligopeptide-modified poly-beta-amino-esters (OM-PBAEs) and related polymers without using DMSO as a solvent yield OM-PBAEs with improved storage stability in biocompatible buffers. The polymers can be stored for extended periods and used to encapsulate nucleic acids and viral vectors losing transfection or transduction efficiency.

### CROSS-REFERENCE TO RELATED APPLICATIONS

This application claims priority to U.S. Provisional Application No. 62/903,799, filed 21 Sep. 2019, which is incorporated by reference herein in its entirety.

### BACKGROUND

Gene therapy offers a novel therapeutic approach for the treatment of a wide range of hereditary or non-hereditary conditions. Several viral and non-viral vectors have been explored in the area of gene therapy. Viral vectors are one of the most popular tools since they are highly efficient in transfection and provide long term gene expression. However, there are significant safety concerns regarding their toxic, immunogenic profile and non-specificity. Non-viral vectors are promising alternatives given their relative safety, ease of production, and modification for specific cell targeting, but their low transfection efficiencies limit their transition into the clinic. Another alternative is nanotechnology mediated solutions that include coating or modification of viruses using polymers which could overcome the obstacles of viral and non-viral systems and increase the overall efficiency through a synergetic effect.

Several polymer systems have been applied in the field of gene therapy, including cationic synthetic polymers, polysaccharides, and polypeptides (Lv, H et al. 2006). Poly (beta-amino ester)s (PBAEs) have been recognized as the most promising candidates in polymeric gene delivery systems in the last decades, thanks to their biodegradable and pH-sensitive features. More than 2000 PBAEs have been synthesized using different diacrylate and amine monomers. Further, end group modifications have been performed using different functional groups including amine, peptide and sugar molecules. Screening of different PBAEs demonstrated that the structure of the terminal group on polymers plays a critical role on the performance and cytotoxicity of gene delivery systems (Zugates, G T, et al., 2007; Green, J J, et al., 2008, Anderson D G, et al., 2005).

Up to the present, most end-modification reactions of PBAEs have been performed in DMSO as reaction solvent. Even if the reaction was not performed in DMSO, polymers were dissolved and stored in DMSO until further use (Green, J J, et al., 2008). DMSO is a commonly used agent to solubilize polar or non-polar drugs in therapeutic applications; however safety concerns arise with the use of DMSO in drug formulations. There is no clear consensus regarding the use of DMSO, but potential cytotoxic effects of DMSO have been documented (de Abreau Costa, et al., 2017, Galvao, J., et al., 2013). More recently, it has also been demonstrated that even low residual concentrations of DMSO can induce undesired effects, so the use of DMSO should be avoided (Verheijem, M, et al., 2019). Therefore, there is a need for methods for the preparation of end-modified PBAEs using DMSO-free conditions.

### SUMMARY

The present technology provides a novel synthesis of oligopeptide end-modified PBAEs and related polymers in DMSO-free conditions. PBAEs synthesized according to the new method were used to coat lentiviral particles to generate a nanoparticle gene delivery system. Further, the polymer-coated virus particles were used in cell transduction experiments. Efficacy and cytotoxicity of the system were evaluated and compared to a system prepared with polymers synthesized conventionally in DMSO. Moreover, the production of OM-PBAE under DMSO-free conditions can be performed at large scale.

OM-PBAEs synthesized according to the present technology can have, for example, a structure as shown in Formula I or II below.

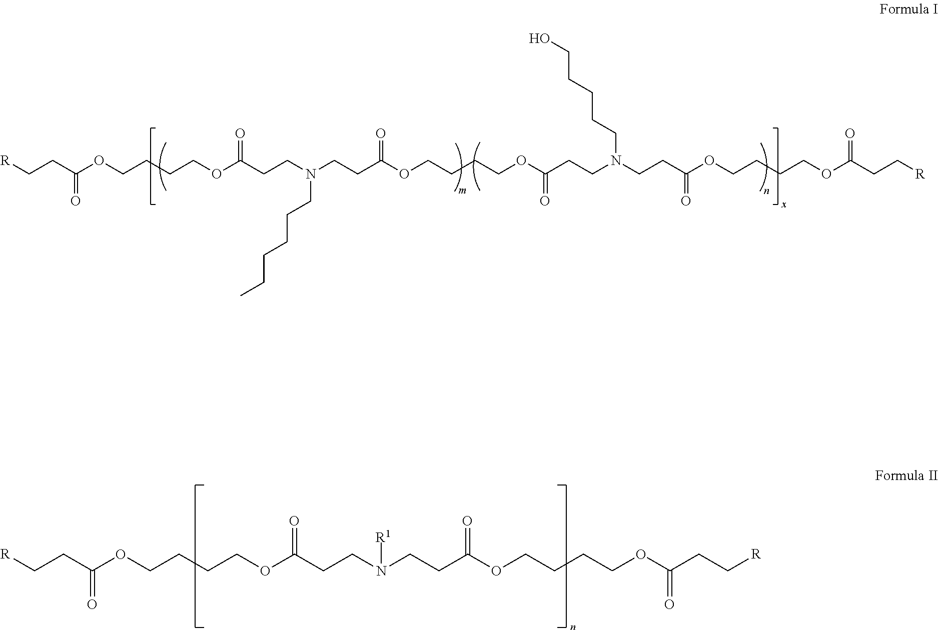

R at the termini represents the same or different oligopeptides, each containing from 2 to 20 amino acid residues; “m” and “n” indicate the number of repeating units with aliphatic or hydroxylated side chains, respectively; “x” indicates the total number of repeating units of aliphatic and hydroxylated blocks in the OM-PBAE.

Other polymers which can be synthesized according to the present technology include those shown in Formula III, Formula IV, and Formula V below.

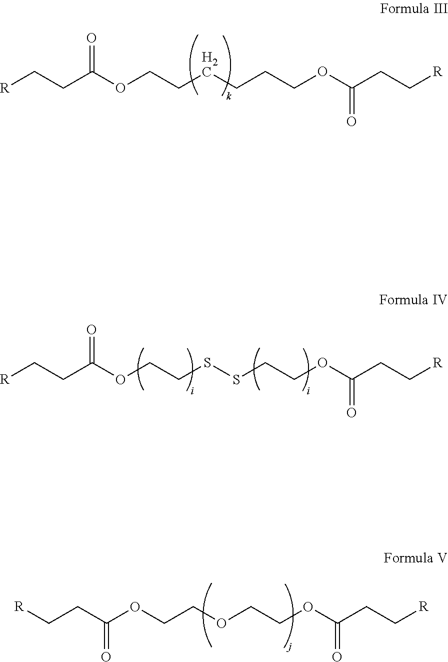

For these polymers, k is an integer from 1 to 50000, and j is an integer from 1 to 20000. R at the termini represents the same or different oligopeptides, each containing from 2 to 20 amino acid residues.

Amino acid residues in the oligopeptides can be any naturally occurring or synthetic amino acids. The amino acid residues can be naturally occurring L-amino acids. The peptides can contain positively charged amino acids, neutral amino acids, hydrophobic amino acids, polar uncharged amino acids, or negatively charged amino acids, or any combination thereof. The oligopeptides can contain a terminal cysteine which can be used to couple the oligopeptide to an acrylate end group of a PBAE acrylate or diacrylate precursor that is reacted with the oligopeptide via a thiol-acrylate Michael addition reaction.

Oligopeptides can have any amino acid sequence. The sequence can contain, for example, an N-terminal cysteine and one or more positively charged amino acids, such as any combination of H, R and K, up to a maximum of 20 amino acid residues. The sequence can contain, for example, an N-terminal cysteine and one or more negatively charged amino acids, such as any combination of D and E, up to a maximum of 20 amino acid residues. The sequence can contain, for example, an N-terminal cysteine and one or more positively charged amino acids, such as H, R or K, combined in any order with any negatively charged amino acids, such as D or E, up to a maximum of 20 amino acid residues. Exemplary amino acid sequences include CH, CHH, CHHH (SEQ ID NO:1), CHHHH (SEQ ID NO:2), CHHHHH (SEQ ID NO:3), CR, CRR, CRRR (SEQ ID NO:4), CRRRR (SEQ ID NO:5), CRRRRR (SEQ ID NO:6). CK, CKK, CKKK (SEQ ID NO:7), CKKKK (SEQ ID NO:8), CKKKKK (SEQ ID NO:9), CE, CEE, CEEE (SEQ ID NO:10), CEEEE (SEQ ID NO:11), CEEEEE (SEQ ID NO:12), CD, CDD, CDDD (SEQ ID NO:13), CDDDD (SEQ ID NO:14), CDDDDD (SEQ ID NO:15), CHRH (SEQ ID NO:16), CHRR (SEQ ID NO:17), CHKH (SEQ ID NO:18), CHKK (SEQ ID NO:19), CHEH (SEQ ID NO:20), CHEE (SEQ ID NO:21), CHDH (SEQ ID NO:22), CHDD (SEQ ID NO:23), CRHR (SEQ ID NO:24), CRHH (SEQ ID NO:25), CRKR (SEQ ID NO:26), CRKK (SEQ ID NO:27), CRER (SEQ ID NO:28), CREE (SEQ ID NO:29), CRDR (SEQ ID NO:30), CRDD (SEQ ID NO:31), CKHK (SEQ ID NO:32), CKHH (SEQ ID NO:33), CKRK (SEQ ID NO:34), CKRR (SEQ ID NO:35), CDHD (SEQ ID NO:36), CDHH (SEQ ID NO:37), CDRD (SEQ ID NO:38), CDRR (SEQ ID NO:39), CDKD (SEQ ID NO:40), CDKK (SEQ ID NO:41), CEHE (SEQ ID NO:42), CEHH (SEQ ID NO:43), CERE (SEQ ID NO:44), CERR (SEQ ID NO:45), CEDE (SEQ ID NO:46), and CEDD (SEQ ID NO:47).

Oligopeptides of the present technology also can be cell penetrating peptides, such as GRKKRRQRRRPQ (TAT) (SEQ ID NO:48), RQIKIWFQNRRMKWKKGG (penetratin) (SEQ ID NO:49), CGYGPKKKRKVGG (NLS sequence) (SEQ ID NO:50), AGYLLGKINLKALAALAKKIL (transportan10) (SEQ ID NO:51), KETWWETWWTEWSQPKKKRRV (pep-1) (SEQ ID NO:52), KLALKLALKALKAALKLA (MAP) (SEQ ID NO:53), RRRRNRTRRNRRRVR (FHV coat) (SEQ ID NO:54), and LLIILRRRIRKQAHAHSK (pVEC) (SEQ ID NO:55). Oligopeptides of the present technology also can be integrin-binding peptides such as RGD or other integrin-binding peptides.

The present technology includes a method for synthesizing an end modified poly-beta-amino-ester (PBAE). The method includes the steps of: (a) providing an end modifier such as an oligopeptide, and a PBAE comprising a terminal acrylate group (PBAE acrylate or diacrylate); (b) forming or providing a first solution containing the PBAE dissolved in acetonitrile; (c) forming or providing a second solution containing the end modifier dissolved in an aqueous citrate solution; and (d) mixing the first and second solutions, whereby the end modifier bonds to the terminal vinyl carbon to form the end modified PBAE.

A PBAE diacrylate for use in the synthesis described above can have a structure, for example, according to Formula VI or Formula VII below.

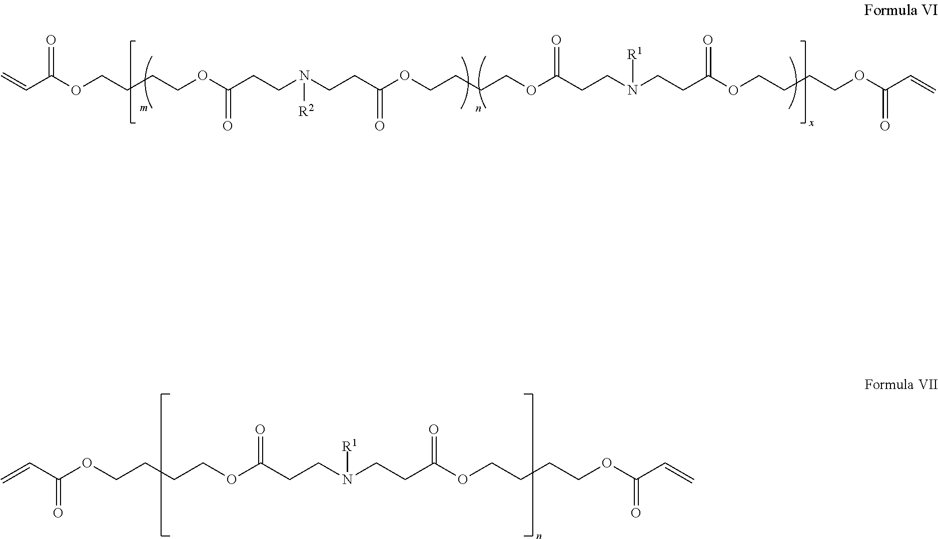

The PBAE diacrylate backbone structure can further be varied by selecting different diacrylate starting material used in the synthesis. For example, the following polymer diacrylates of Formula VIII, Formula IX, or Formula X can be used in the synthesis:

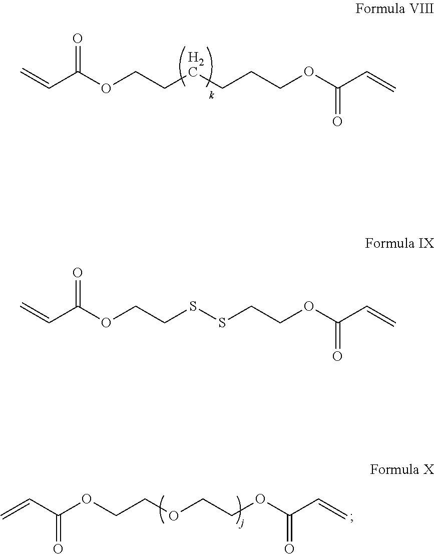

wherein R¹ and R² are each independently selected from the first group consisting of hydrogen, halogen, alkyl, alkenyl, alkynyl, phenyl, cycloalkyl, cycloalkenyl, cycloalkynyl, aryl, and heteroaryl; wherein for R¹ and R² each independently one or more carbons may be substituted by O, N, B, or S; wherein independently each constituent of the first group can optionally be further substituted with one or more substituents selected from the second group consisting of —OH, halogen, acyl halide, carbonate, ketone, aldehyde, ester, methoxy, ether, amide, amine, nitrile, and any other constituent of the first group; wherein R¹ and R² each independently have at most 20 total carbons; wherein n and m independently are integers from 2 to 10000; k is an integer from 1 to 50000; j is an integer from 1 to 20000; and wherein X is an integer from 1 to 5000.

As used herein, “halogens” are elements selected from fluorine, chlorine, bromine, and iodine. “Alkyl” groups can be unbranched or branched and can optionally be added as a substituent to a molecular structure by replacement of any hydrogen atom. The bonded alkyl chain atom may be carbon, or may be O, N, B, or S, if the alkyl contains one or more heteroatoms.

The present technology also includes a method of purifying a PBAE-diacrylate polymer. The method includes the following steps: (a) dissolving the PBAE-diacrylate polymer in ethyl acetate; (b) precipitating the PBAE-diacrylate polymer by adding dropwise into heptane to yield a ratio of heptane to ethyl acetate of about 10/1 volume/volume; and (c) repeating steps (a) and (b) twice, whereby the purified PBAE-diacrylate polymer is obtained.

The present technology also includes another method of purifying a PBAE-diacrylate polymer. The method includes the following steps: (a) dissolving the PBAE-diacrylate polymer in ethyl acetate; and (b) precipitating the PBAE-diacrylate polymer from the solution obtained in step (a) by adding heptane to the solution to yield a ratio of heptane to ethyl acetate of about 2/1 volume/volume, whereby the purified PBAE-diacrylate polymer is obtained as the precipitate.

The present technology also includes a method of purifying an oligopeptide-modified PBAE (OM-PBAE). The method includes the following steps: (a) extracting the OM-PBAE with ethanol, and then drying the extracted OM-PBAE; (b) re-dissolving the OM-PBAE resulting from step (a) in ethanol, and precipitating the OM-PBAE in diethylether/acetone at a ratio of about 7/3 (v/v); (c) washing the precipitate resulting from step (b) with diethylether/acetone (about 7/3 v/v); and (d) removing residual solvents from the OM-PBAE resulting from step (c).

The present technology also includes a method of purifying an oligopeptide-modified PBAE (OM-PBAE). The method includes the following steps: (a) passing the OM-PBAE through a size exclusion column using an eluent comprising water; (b) collecting the OM-PBAE after passing through the size exclusion column; and (c) removing residual solvents from the OM-PBAE resulting from step (b).

The present technology can be further summarized by the following features:

1. A method for synthesizing an end modified polymer, the method comprising:

(a) providing an end modifier and a polymer comprising a terminal acrylate group comprising a terminal vinyl carbon;

(b) forming a first solution comprising the polymer dissolved in acetonitrile;

(c) forming a second solution comprising the end modifier dissolved in an aqueous citrate solution; and

(d) mixing the first and second solutions, whereby the end modifier bonds to the terminal vinyl carbon to form the end modified polymer.

2. The method of feature 1, wherein the second solution further comprises acetonitrile.

3. The method of any of feature 2, wherein the second solution is formed by mixing the end modifier with an aqueous citrate solution until the end modifier is dissolved in the solution, then adding acetonitrile to the solution.

4. The method of feature 2 or feature 3, wherein the second solution comprises water/acetonitrile in a ratio from about 1/1 volume/volume to about 2/1 volume/volume.

5. The method of any of the preceding features, wherein the second solution comprises about 25 mM citrate and has a pH of about pH 5.0.

6. The method of any of the preceding features, wherein the mixing of the first and second solutions in step (d) forms a solution comprising acetonitrile/water at a ratio of about 3/2 volume/volume.

7. The method of any of the preceding features, wherein the end modifier comprises a thiol and the end modifier bonds to the terminal vinyl carbon through a thioether bond (—C—S—C—).

8. The method of any of the preceding features, wherein the end modifier is an oligopeptide selected from the group consisting of CRRR (SEQ ID NO:4), CKKK (SEQ ID NO:7), CHHH (SEQ ID NO:1), CDDD (SEQ ID NO:13), CEEE (SEQ ID NO:10), GRKKRRQRRRPQ (TAT) (SEQ ID NO:48), RQIKIWFQNRRMKWKKGG (penetratin) (SEQ ID NO:49), CGYGPKKKRKVGG (NLS, in-nuclear translocation sequence of SV-40 large T-antigen) (SEQ ID NO:50), AGYLLGKINLKALAALAKKIL (transportan10) (SEQ ID NO:51), RGD, KETWWETWWTEWSQPKKKRRV (pep-1) (SEQ ID NO:52), KLALKLALKALKAALKLA (MAP) (SEQ ID NO:53), RRRRNRTRRNRRRVR (FHV coat) (SEQ ID NO:54), and LLIILRRRIRKQAHAHSK (pVEC) (SEQ ID NO:55).

9. The method of any of the preceding features, wherein the end modifier is selected from the group consisting of cysteine, homocysteine, and oligopeptides comprising cysteine or homocysteine, wherein the oligopeptide contains not more than 20 amino acids.

10. The method of feature 8 or feature 9, wherein the mixing of the first and second solutions forms a solution comprising thiol/acrylate at a molar ratio in the range from about 2.2/1 to about 3/1.

11. The method of feature 8 or feature 9, wherein the oligopeptide is provided as a hydrochloride salt, an acetate salt, a TFA salt, a formate salt, or a combination thereof.

12. The method of any of the preceding features, wherein both the first solution and the second solution do not contain DMSO.

13. The method of any of the preceding features, wherein the mixing of the first and second solutions in step (d) is carried out in an inert atmosphere, wherein the inert atmosphere reduces formation of di-sulfide during the mixing.

14. The method of any of the preceding features, wherein the mixing of the first and second solutions in step (d) is carried out for about 20 hours.

15. The method of any of the preceding features, wherein the mixing the first and second solutions in step (d) is carried out at about 25° C.

16. The method of any of the preceding features, wherein (i) the PBAE comprising a terminal acrylate group has a structure of Formula VI or Formula VII:

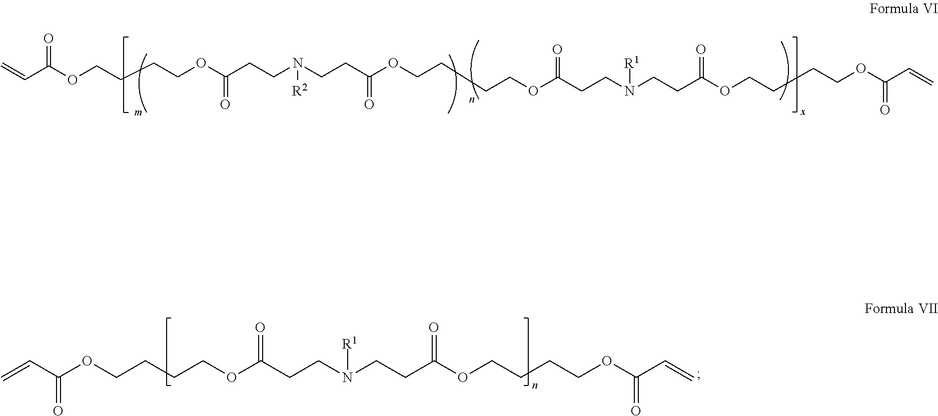

or wherein (ii) the polymer comprising a terminal acrylate group has a structure of Formula VIII, Formula IX, or Formula X:

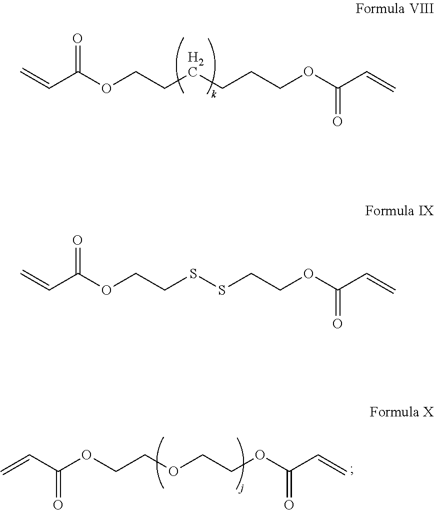

wherein R1 and R2 are each independently selected from the first group consisting of hydrogen, halogen, alkyl, alkenyl, alkynyl, phenyl, cycloalkyl, cycloalkenyl, cycloalkynyl, aryl, and heteroaryl; wherein for R1 and R2 each independently one or more carbons may be substituted by O, N, B, or S; wherein independently each constituent of the first group can optionally be further substituted with one or more substituents selected from the second group consisting of —OH, halogen, acyl halide, carbonate, ketone, aldehyde, ester, methoxy, ether, amide, amine, nitrile, and any other constituent of the first group; wherein R1 and R2 each independently have at most 20 total carbons;

wherein n and m independently are integers from 2 to 10000; k is an integer from 1 to 50000; j is an integer from 1 to 20000; and wherein X is an integer from 1 to 5000.

17. The method of any of the preceding features, further comprising:

(e) removing residual solvents from the end modified polymer obtained in step (d).

18. The method of feature 17, wherein the resulting end modified polymer obtained in step (e) comprises residual citrate.

19. A composition comprising an end modified polymer made by the method of any one of the preceding features.

20. The composition of feature 19, wherein the composition is essentially free of DMSO.

21. The composition of feature 19 or feature 20 which is an aqueous solution, wherein the end modified PBAE has a half-life of at least 10 weeks when the composition is stored at about −20° C.

22. The composition of feature 21, wherein the end modified PBAE has a half-life of at least 15 weeks.

23. A method of purifying a polymer diacrylate, the method comprising:
(a) dissolving the polymer diacrylate in ethyl acetate;
(b) precipitating the polymer diacrylate by adding dropwise into heptane to yield a ratio of heptane to ethyl acetate of about 10/1 volume/volume; and
(c) repeating steps (a) and (b) twice, whereby the purified polymer diacrylate is obtained.

24. A method of purifying a polymer diacrylate, the method comprising:
(a) dissolving the polymer diacrylate in ethyl acetate; and
(b) precipitating the polymer diacrylate from the solution obtained in step (a) by adding heptane to the solution to yield a ratio of heptane to ethyl acetate of about 2/1 volume/volume, whereby the purified polymer diacrylate is obtained as the precipitate.

25. The method of feature 24, further comprising performing the method of feature 25 using the product of the method of feature 24 as the starting polymer diacrylate of feature 25.

26. A purified polymer diacrylate obtained by the method of any of features 23-25.

27. A method of purifying an oligopeptide-modified PBAE (OM-PBAE), the method comprising:
(a) extracting the OM-PBAE with ethanol, and then drying the extracted OM-PBAE;
(b) re-dissolving the OM-PBAE resulting from step (a) in ethanol, and precipitating the OM-PBAE in diethylether/acetone at a ratio of about 7/3 (v/v);
(c) washing the precipitate resulting from step (b) with diethylether/acetone (about 7/3 v/v); and
(d) removing residual solvents from the OM-PBAE resulting from step (c).

28. A method of purifying an oligopeptide-modified PBAE (OM-PBAE), the method comprising:
(a) passing the OM-PBAE through a size exclusion column using an eluent comprising water;
(b) collecting the OM-PBAE after passing through the size exclusion column; and
(c) removing residual solvents from the OM-PBAE resulting from step (b).

29. The method of feature 27 or feature 28, wherein step (c) comprises applying vacuum or performing lyophilization, precipitation, filtration, centrifugation, washing the OM-PBAE with diethylether/acetone, or a combination thereof.

30. An OM-PBAE obtained by the method of any of features 19-22 or 27-29.

31. A nanoparticle comprising a nucleic acid or a viral vector encapsulated with the OM-PBAE of feature 30.

32. The nanoparticle of feature 31, wherein the viral vector is a lentiviral vector.

33. The nanoparticle of feature 32, wherein the nanoparticle has a higher transduction efficiency compared to a nanoparticle comprising an OM-PBAE made by a method comprising the use of DMSO as solvent.

34. The nanoparticle of feature 32, wherein the nanoparticle is capable of transducing cells yielding a higher cell viability compared to a nanoparticle comprising an OM-PBAE made by a method comprising the use of DMSO as solvent.

As used herein, the term “about” includes values within 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of”.

### BRIEF DESCRIPTION OF DRAWINGS

FIG. 1 shows results of solvent screening for solubility of PBAE-diacrylate, peptide-HCl salts, and OM-PBAEs in common organic solvents and solvent mixtures (working concentrations ca. 10-20 mg/mL). CIT=25 mM citrate buffer pH 5.0. Shading pattern X=soluble, black=not soluble, white=not tested. The combinations indicated by solid grey were deemed to be insoluble during solubility testing (within 1 hour) but were later observed to be soluble during reactions and work-up. It is unclear whether this was due to a difference in dissolution time, concentration, solid surface area, and/or presence of other solutes.

FIG. 2 shows a thin layer chromatography plate spotted with crude PBAE-diacrylate (lane 1) obtained from classical protocol, purified PBAE-diacrylate by 1/10, ethylacetate/heptane (lane 2), and purified PBAE-diacrylate by 1/2, ethyl acetate/heptane (lane 3) obtained from the novel synthesis protocol described herein. Dichloromethane/methanol (12/1, v/v) was used as mobile phase, and staining was by KMnO₄.

FIGS. 3A and 3B show the GPC traces of crude PBAE-diacrylate obtained by the classical method (3A) and purified PBAE-diacrylate polymer obtained via the new synthesis protocol described herein (3B).

FIGS. 4A and 4B show physical appearance tests performed with solutions of crude and purified PBAE-diacrylate polymers in citrate buffer (25 mM, pH 5.0) at t=0 (4A) and t=20 h (4B).

FIG. 5 shows an NMR spectrum of PBAE-CR3 synthesized in acetonitrile/citrate (25 mM, pH 5.0) (3/2, v/v) using crude PBAE-diacrylate as starting material. The ratio of the integration values of acrylate to CH₃ peaks was used to calculate the acrylate conversion.

FIG. 6 shows an NMR spectrum of PBAE-CR3 synthesized in acetonitrile/citrate (25 mM, pH 5.0) (3/2, v/v) using purified PBAE-diacrylate as starting material and two times concentrated peptide solution. The ratio of the integration values of acrylate to CH₃ peaks was used to calculate the acrylate conversion.

FIGS. 7A and 7B provide an overview of the OM-PBAE synthesis and purification steps in the classical protocol (prior art) (7A) and the new DMSO-free method of the present technology (7B).

FIGS. 8A, 8B, and 8C-8J show in vitro results obtained with frozen human lymphocyte preparations transduced with lentivectors encoding Green Fluorescent Protein (GFP) and coated with PBAE-CR3 in DMSO obtained with crude PBAE-diacrylate (Entry 3); PBAE-CH3 obtained with crude PBAE-diacrylate (Entry 2); 60/40 or 40/60 mixes of PBAE-CR3/PBAE-CH3 obtained with crude PBAE-diacrylate; PBAE-CR3 obtained with purified PBAE-diacrylate (Entry 8); PBAE-CH3 obtained with crude PBAE-diacrylate (Entry 7); 60/40 or 40/60 mixes of PBAE-CR3/PBAE-CH3 obtained with purified PBAE-diacrylate. In FIG. 8A the transduction efficiency is given for each tested condition. FIG. 8B shows cell viability 72 h post-transduction of cells. Lymphocytes populations transduced with the different polymer-coated lentiviral vectors and analyzed by flow cytometry staining with T (CD3⁺) and B (CD19⁺) specific antibodies are compared in FIG. 8C to FIG. 8J. Controls include cells that have not been transduced (NT) but kept in culture throughout the experiment and cells transduced with the non-encapsulated VSV-G(−) (“Bald”) Lentiviral Vector Particles (LV).

FIGS. 9A and 9B show transduction efficiency (9A) and cell viability (9B) results of an experiment similar to that shown in FIGS. 8A-8B but carried out on freshly isolated human PBMCs.

FIGS. 10A and 10B show transduction efficiency (10A) and cell viability (10B) results of an experiment similar to that shown in FIGS. 8A-8B but carried out on freshly isolated human PBMCs transduced with lentivectors encoding GFP and coated with a 100/0, 60/40, 40/60, or 0/100 mix of PBAE-CR3 (Entry 3)/PBAE-CH3 (Entry 2), as indicated, in DMSO obtained with crude PBAE-diacrylate (left bars, dark grey), in DMSO-free PBAE-CR3/PBAE-CH3 obtained with crude PBAE-diacrylate and without post-coupling purification (center bars, light grey), or in DMSO-free PBAE-CR3 (Entry 13)/PBAE-CH3 (Entry 12) obtained with crude PBAE-diacrylate and post-coupling purification (right bars, white).

FIGS. 11A and 11B show transduction efficiency (11A) and cell viability (11B) results of an experiment similar to that shown in FIGS. 8A-8B but carried out on freshly isolated human PBMCs transduced with lentivectors encoding GFP and coated with a 60/40 mix of PBAE-CR3 (Entry 3)/PBAE-CH3 (Entry 2) in DMSO obtained with crude PBAE-diacrylate; 60/40 mix of DMSO-free PBAE-CR3 (Entry 13)/PBAE-CH3 (Entry 12) obtained with crude PBAE-diacrylate and post-coupling purification. Biological properties of polymers prepared with the new synthesis protocol of the present technology and freshly resuspended in water or stored for 5 or 15 weeks in water at −20° C. are compared with polymers prepared in DMSO with the classical protocol.

FIGS. 12A and 12B show transduction efficiency (12A) and cell viability (12B) results of an experiment similar to that shown in FIGS. 8A-8B but carried out on freshly isolated Human PBMCs transduced with lentivectors encoding GFP and coated with a 60/40 mix of PBAE-CR3 (Entry 3)/PBAE-CH3 (Entry 2) in DMSO obtained with crude PBAE-diacrylate (grey bar); 60/40 mix of DMSO-free PBAE-CR3 (Entry 13)/PBAE-CH3 (Entry 12) obtained with crude PBAE-diacrylate and post-coupling purification (third bar from right); 60/40 mix of DMSO-free PBAE-CR3 (Entry 16)/PBAE-CH3 (Entry 22) obtained with purified PBAE-diacrylate without post-coupling purification (second bar from right); 60/40 mix of DMSO-free PBAE-CR3 (Entry 17)/PBAE-CH3 (Entry 23) obtained with purified PBAE-diacrylate and post-coupling purification (far right bar).

FIGS. 13A and 13B show transduction efficiency (13A) and cell viability (13B) results of an experiment similar to that shown in FIGS. 8A-8B but carried out with lentivectors encoding GFP and coated with a 60/40 mix of PBAE-CR3 (Entry 3)/PBAE-CH3 (Entry 2) in DMSO obtained with crude PBAE-diacrylate; 60/40 mix of DMSO-free PBAE-CR3 (Entry 17)/PBAE-CH3 (Entry 23) obtained with purified PBAE-diacrylate and post-coupling purification. Time zero storage in DMSO-free buffer is indicated by the bars at left; time 2 weeks at −20° C. in DMSO-free buffer is indicated by the center bars; and time 10 weeks at −20° C. in DMSO-free buffer is indicated by the bars at right for each condition. Biological properties of polymers prepared with new synthesis protocol and freshly resuspended in water or stored for 2 or 10 weeks in water at −20° C. are compared with polymers prepared in DMSO with classical protocol.

FIGS. 14A and 14B show transduction efficiency (14A) and cell viability (14B) results of an experiment similar to that shown in FIGS. 8A-8B but carried out with lentivectors encoding GFP and coated with a 60/40 mix of PBAE-CR3 (Entry 3)/PBAE-CH3 (Entry 2) in DMSO obtained with crude PBAE-diacrylate; 60/40 mix of DMSO-free PBAE-CR3 (Entry 17)/PBAE-CH3 (Entry 23) obtained with purified PBAE-diacrylate and post-coupling purification; 60/40 mix of DMSO-free PBAE-CR3 (Entry 18)/PBAE-CH3 (Entry 23) obtained with purified PBAE-diacrylate, using a 2-fold concentrated peptide solution for the coupling reaction and post-coupling purification; 60/40 mix of DMSO-free PBAE-CR3 (Entry 19)/PBAE-CH3 (Entry 23) obtained with purified PBAE-diacrylate, using a 2-fold concentrated CRRR peptide solution for the coupling reaction and post-coupling purification with ethanol extraction repeated 3 times and dissolution of dried ethanol extracts in ethanol; 60/40 mix of DMSO-free PBAE-CR3 (Entry 20)/PBAE-CH3 (Entry 23) obtained with purified PBAE-diacrylate, using a 2-fold concentrated CRRR peptide solution for the coupling reaction and post-coupling purification with ethanol extraction repeated twice and dissolution of dried ethanol extract in ethanol/water (4/1, v/v); 60/40 mix of DMSO-free PBAE-CR3 (Entry 21)/PBAE-CH3 (Entry 23) obtained with purified PBAE-diacrylate, using a 2-fold concentrated CRRR peptide solution for the coupling reaction and post-coupling purification with ethanol extraction repeated twice and dissolution of dried ethanol extract in in ethanol/water (3/2, v/v).

### DETAILED DESCRIPTION

The present technology provides methods for synthesizing end-modified PBAEs, or oligopeptide-modified PBAEs (OM-PBAEs) without the use of DMSO in the reaction as solvent. The technology also provides compositions containing the synthesized polymers, methods utilizing the compositions, and purification methods for reactants and products of the synthesis.

A method for DMSO-free synthesis of OM-PBAEs can include dissolving a PBAE polymer having at least one terminal acrylate group in a solvent or mixture of solvents. An end modifier, such as an oligopeptide, can be dissolved in the same solvent or solvent mixture, or can be provided in a separate solution to be mixed with the solubilized polymer. After the end modifier is in a reaction solution with the polymer for a reaction time, at least a portion of the end modifier will react with the terminal vinyl carbon of the polymer, to form the end-modified PBAE or OM-PBAE. The reaction can be carried out for any suitable reaction time, at any suitable reaction temperature and pressure. Other suitable reaction conditions can be selected as desired, including use of a catalyst, application of electromagnetic radiation (e.g. UV/visible light, microwaves), sonic mixing, stirring, pH, reflux, or any combination thereof. The reaction can be through a Michael addition or other reaction mechanism.

The starting PBAE polymer, including at least one terminal acrylate group, and the end modifier should be at least partially soluble in the solvent or solvent mixture chosen for the reaction method. As described below, any soluble polymer with a reactive end vinyl or terminal acrylate group can be applied to the methods, along with an end modifier that has a suitable nucleophile therein. For example, without intending to limit the present technology to any particular mechanism, the reactions herein can provide nucleophilic attack on the end terminal carbon of a vinyl group to form a bond in a synthesis step. Other conditions can be used before or after this step. The kinetics of the reaction can be altered by utilizing, for example, a gel or viscous solvent condition, steric effects, temperature, substrates, particles, or additives. In another example, a polymer or PBAE including a terminal acrylate group can be provided with a protecting group or with a binding to a fixed substrate or to particles, to selectively bind the end modifier to one end of the polymer or PBAE that is not bound to the protecting group, fixed substrate, or particles. As such, steric effects can be included in the methods herein.

The end modifiers can be oligopeptides. The oligopeptides can react with the end terminal carbon of a vinyl group with a reduction of the end terminal carbon. An oligopeptide can include a nucleophilic carbon, sulfur, nitrogen, oxygen, or other atom. The nucleophile can be on a terminal end of the oligopeptides, for example, as a thiol on a cysteine. Determination of which nucleophile bonds to the acceptor (terminal vinyl carbon) can be changed by selected reaction conditions.

The methods of making the end modified PBAEs can include a one-step synthetic strategy, wherein at least a portion of polymer or PBAE including a terminal acrylate group is converted to an end modified PBAE, a reaction product, in a single synthesis step. For example, a solution of polymer may be at least partially converted to an end modified PBAE in a single synthesis step herein. A method for synthesizing an end modified PBAE can include providing an end modifier and a polymer or PBAE including a terminal acrylate group and including a terminal vinyl carbon; and forming a solution with the PBAE and the end modifier dissolved in solution, whereby the end modifier bonds to the terminal vinyl carbon to form the end modified PBAE. The solution can be a mixture of acetonitrile and an aqueous citrate solution, or other suitable solvent or solvent mixture. Preferably, the solvent or solvent mixture does not include dimethyl sulfoxide (DMSO). Preferably, the solvent or solvent mixture contains less than 10%, less than 5%, less than 2%, less than 1%, or less than 0.1%, or even less than 0.01% DMSO, or 0% DMSO, on a volume or weight basis. After the solution is formed, the one-step synthesis can be carried out for a period of time with or without mixing or other conditions. After formation of the end-modified PBAE, isolation or purification of the end-modified PBAE can be performed by any known means.

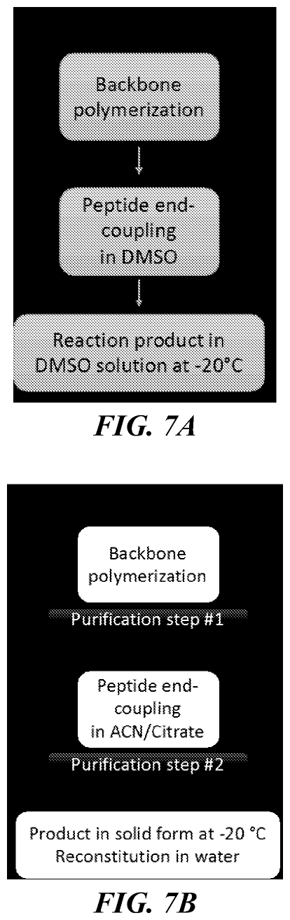

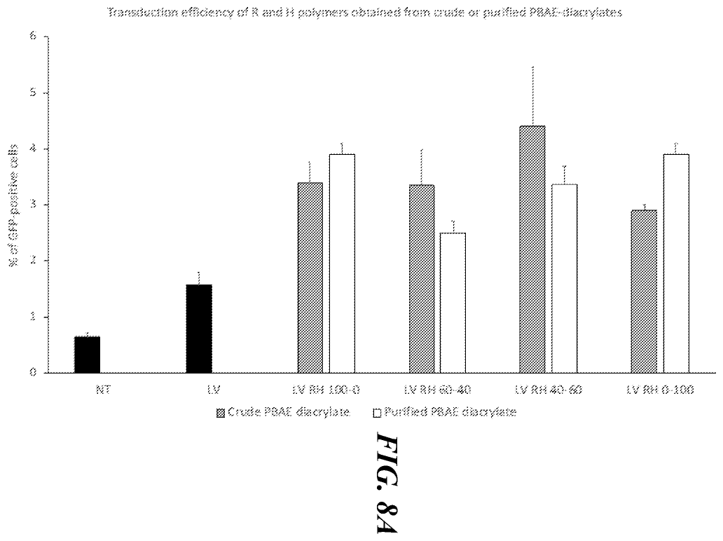

FIG. 7A illustrates the classical method for synthesis of peptide end coupled PBAEs, which is performed using DMSO as solvent. At the top of FIG. 7A, a polymer is formed by backbone polymerization. At the middle of FIG. 7A, peptide end coupling is accomplished in DMSO, and at the bottom of FIG. 7A, the reaction product is stored in DMSO at −20° C. In FIG. 7B, the present method for DMSO-free synthesis of peptide end coupled PBAEs is shown. The purification steps shown in black background are optional. At the center of FIG. 7B, the peptide end coupling is performed in DMSO-free reaction conditions. The use of an acetonitrile/citrate solvent mixture (ACN/citrate) can provide synthesis of oligopeptide modified PBAEs (OM-PBAEs) entirely free of DMSO.

The synthetic methods described herein can be carried out in more than one step. The starting materials, including a PBAE having a terminal acrylate group and the end modifier, can be provided as salts, for example, to aid solubility. The starting materials can be provided in separate solutions and combined to form a one-step synthetic strategy. Purification, storage, or other methods carried out after synthesis can be further beneficial, for example, to reduce toxicity, increase storage stability, or to preserve or increase transfection or transduction efficiency.

Any of the methods or compositions disclosed herein can be provided in or stored in any salt form, any crystal form, any co-crystal form, an amorphous form, any polymorph form, or a combination thereof.

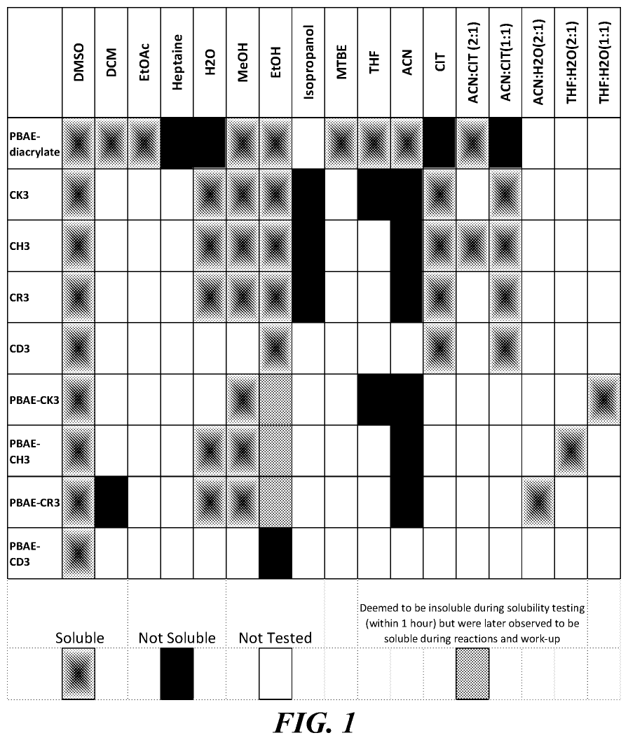

A solubility comparison of different solvents, solvent mixtures, or ratios of different solvents, can be conducted so as to provide effective solubilization of reactants and products for the synthesis to occur. An example of a solubility comparison is provided in FIG. 1 (Example 2). Starting materials such as PBAE diacrylates and end modifiers, with or without salts, can be tested. Reaction products can be tested. Solvents or solvent mixtures with lower solubility can provide slower reaction conditions. Kinetics can be modified as desired. As discussed below in Example 2, ethanol as a reaction solvent can provide a lowered reaction rate. Methanol can provide a reduced molecular weight.

Any of the methods or compositions disclosed herein can be carried out in, or reactants or products stored in, an inert atmosphere. An inert atmosphere can prevent formation of side products, impurities, or unwanted disulfide bonds. Inert atmospheres can be any atmosphere that prevents an undesired outcome, for example, a vacuum, nitrogen, argon, helium, krypton, xenon, and radon.

An example of a solvent that can dissolve a PBAE with a terminal acrylate group, and that also can dissolve an oligopeptide, is acetonitrile (ACN) combined with an aqueous citrate solution (citrate buffer). The citrate buffer can be at any desired concentration; for example, it can contain about 25 millimolar citrate and have a pH of about 5.0. The ratio of ACN/citrate buffer, as measured by volume before combining, can be any desired ratio; for example, it can be from about 1:1 to about 2:1. The ratio can be about 1.25:1, about 1.5:1, about 1.75:1, or about 2:1. The ratio can be about 3:2. The PBAE acrylate can be dissolved in ACN and the end modifier can be dissolved in citrate buffer, and then the two solutions can be combined. Additional ACN optionally can be added to the solution of end modifier in citrate buffer before combining it with the solution of PBAE acrylate. The amount optionally added ACN can be about (volume water:volume ACN) 1:0.5, about 1:1, about 1.5:1, about 2:1, or about 2.5:1; the desired amount of added ACN can depend upon, for example, temperature or pH. After the starting materials are admixed, other operations such as mixing, stirring, temperature change or other change of conditions can be utilized. The end modifier can include a thiol functional group, and the reaction can form a C—S—C bond.

The starting materials for the reaction can be purified before the reaction by any known method. For example, a PBAE-diacrylate polymer can be purified by dissolution in ethyl acetate and precipitation in heptane. The PBAE-diacrylate polymer or the end modifier can be purified by chromatography, such as reverse phase, normal phase, flash, ion-exchange, hydrophobic interaction, gel filtration, size exclusion, or hydrophilic interaction (HILIC) chromatography, or by dialysis, lyophilization, precipitation, centrifugation, fractionation, or sedimentation.

### EXAMPLES

### Example 1. Synthesis, Purification and Characterization of OM-PBAE in DMSO—Classical Method

Poly (β-amino ester)s (PBAEs) were prepared in a two-step procedure as described by Dosta et al. with slight modifications. First step is the synthesis of PBAE-diacrylate polymers, and the second step includes the synthesis of peptide modified PBAEs (OM-PBAE) in DMSO.

Synthesis, Purification and Characterization of PBAE-diacrylate Polymers

Poly ((3-amino ester)-diacrylate polymer was synthesized via addition type polymerization using primary amine and diacrylate functional monomers. 5-amino-1-pentanol (Sigma-Aldrich, 95.7% purity, 3.9 g, 36.2 mmol), 1-Hexylamine (Sigma-Aldrich, 99.9 purity, 3.8 g, 38 mmol) and 1,4-butanediol diacrylate (Sigma-Aldrich, 89.1% purity, 18 g, 81 mmol) were mixed in a round bottom flask at molar ratio of 2.2:1, acrylate to primary amine groups. The mixture was stirred at 90° C. for 20 h. Then the crude product, a light-yellow viscous oil, was obtained by cooling the reaction mixture to room temperature and stored at −20° C. until further use.

Synthesized PBAE-diacrylate polymers were characterized using 1H-NMR spectroscopy to confirm the structures and GPC to determine the molecular weight characteristics. NMR spectra were collected in Bruker 400 MHz Avance III NMR spectrometer, with 5 mm PABBO BB Probe, Bruker and DMSO-d6 was used as deuterated solvent. Molecular weight determination was conducted on a Waters HPLC system equipped with a GPC SHODEX KF-603 column (6.0×about 150 mm), and THF as mobile phase and with an RI detector. The molecular weights were determined using a conventional calibration curve obtained by polystyrene standards. Weight average molecular weight (M_{w}) and number average molecular weight (Mₙ) of crude PBAE-diacrylate polymer were determined as 4900 g/mol and 2900 g/mol, respectively.

Synthesis, Purification and Characterization of OM-PBAEs in DMSO

OM-PBAE polymers were obtained by peptide end-modification of PBAE-diacrylate polymers via thiol-acrylate Michael addition reaction in DMSO at a thiol/diacrylate ratio of 2.8:1. Synthesis of tri-arginine modified PBAE polymer (PBAE-CR3) is given as an example: crude PBAE-diacrylate polymer (199 mg, 0.08 mmol) was dissolved in DMSO (1.1 mL) and a hydrochloride salt of NH₂-Cys-Arg-Arg-Arg-COOH peptide (SEQ ID NO:4) (CR3—95% purity—purchased from Ontores Biotechnologies, Zhejiang, China) (168 mg, 0.23 mmol) was dissolved in DMSO (1 mL). Then the solutions of polymer and peptide were mixed and stirred at 25° C. in a temperature-controlled water bath for 20 h. Peptide modified PBAE was precipitated in 20 mL diethylether/acetone (7/3, v/v), then the product was washed two times with 7.5 mL diethylether/acetone (7/3, v/v), followed by vacuum drying and resulting product was resuspended in DMSO at a concentration of 100 mg/mL and stored at −20° C. for further use.

In a further example, tri-lysine end-modified PBAE polymer was obtained by mixing a solution of crude PBAE-diacrylate (199 mg, 0.08 mmol) dissolved in DMSO (1.1 mL) and a solution of hydrochloride salt of NH₂-Cys-Lys-Lys-Lys-COOH (SEQ ID NO:7) (CK3) (149 mg, 0.23 mmol) in DMSO (1 mL) and purification step was followed as previously described for PBAE-CR3. For the tri-histidine end-modified PBAE polymer, PBAE-CH3, the solution of PBAE-diacrylate (199 mg, 0.08 mmol) dissolved in DMSO (1.1 mL) and mixed with a solution of hydrochloride salt of NH₂-Cys-His-His-His-COOH (SEQ ID NO:1) (CH3) (154 mg, 0.23 mmol) in DMSO (1.0 mL). For the tri-aspartate end-modified PBAE polymer, PBAE-CD3, the solution of PBAE-diacrylate (199 mg, 0.08 mmol) in DMSO (1.1 mL) was mixed with a solution of hydrochloride salt of NH₂-Cys-Asp-Asp-Asp-COOH (SEQ ID NO:13) (CD3) (114 mg, 0.23 mmol) in DMSO (1 mL). Finally, for the tri-glutamate end-modified PBAE polymer, PBAE-CE3 the solution of PBAE-diacrylate (199 mg, 0.08 mmol) in DMSO (1.1 mL) was mixed with a solution of hydrochloride salt of NH₂-Cys-Glu-Glu-Glu-COOH (SEQ ID NO:10) (CE3) (124 mg, 0.23 mmol) in DMSO (1 mL).

1H-NMR analysis confirmed the expected structures. Further, the percentage of acrylate conversion was determined from the ratio of CH₃ protons (0.8 ppm) on the polymer backbone which was calibrated to the same value as in the spectrum of the starting material to residual acrylate peaks (5.75-6.5 ppm). Therefore, dividing the integration value of acrylate peaks to six (which is the number of protons on the acrylate groups) yielded the residual acrylate amount. The Michael addition reaction efficiencies for peptide modified PBAEs from crude PBAE-diacrylate were determined as; PBAE-CR3: 84%, PBAE-CK3: 93%, PBAE-CH3: 93%, PBAE-CD3: 96% and PBAE-CE3: 98%. However, in all cases overall yields of the reactions were greater than 100% indicating the presence of a large excess of residual DMSO (Table 1). Furthermore, the residual peptide content in each peptide modified PBAE was quantified by UV detection (wavelength 220 nm) after separation by UPLC ACQUITY system (Waters) equipped with a BEH C18 column (130 A, 1.7 μm, 2.1×50 mm, temperature 35° C.) using an acetonitrile/water with 0.1% TFA as gradient.

### Example 2. New Synthesis of OM-PBAE, Purification and Characterization

Implementation of a Purification Step for PBAE-diacrylate Polymers

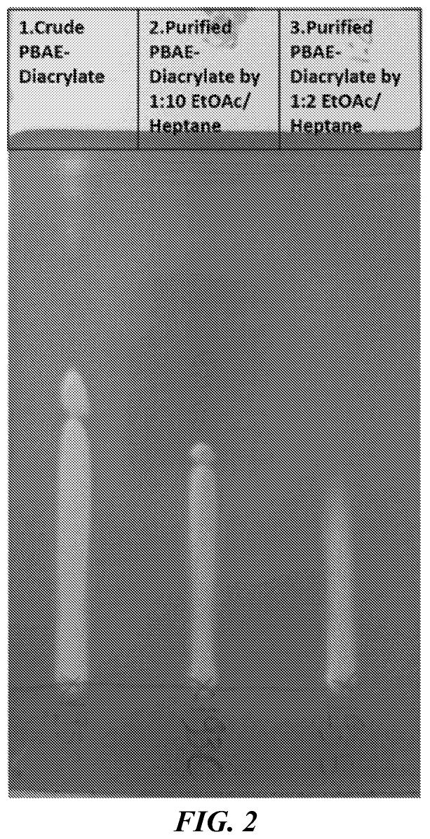

After the synthesis of PBAE-diacrylate polymers as described in Example 1, the reaction was monitored by Thin Layer Chromatography (TLC) using dichloromethane/methanol (12/1, v/v) as mobile phase. Then, the full consumption of amine functional monomers during the polymerization was determined by ninhydrin staining. Additional staining with potassium permanganate (KMnO₄) demonstrated the presence of apolar impurities in the crude product from classical synthesis (FIG. 2, lane 1). In all scientific and technical reports related to PBAEs, crude product was used as obtained without further purification. In the current example, the performance of the purification of PBAE-diacrylate polymers was studied and further studied were the effects of purified PBAE-diacrylate on the synthesis of OM-PBAEs and biological activity.

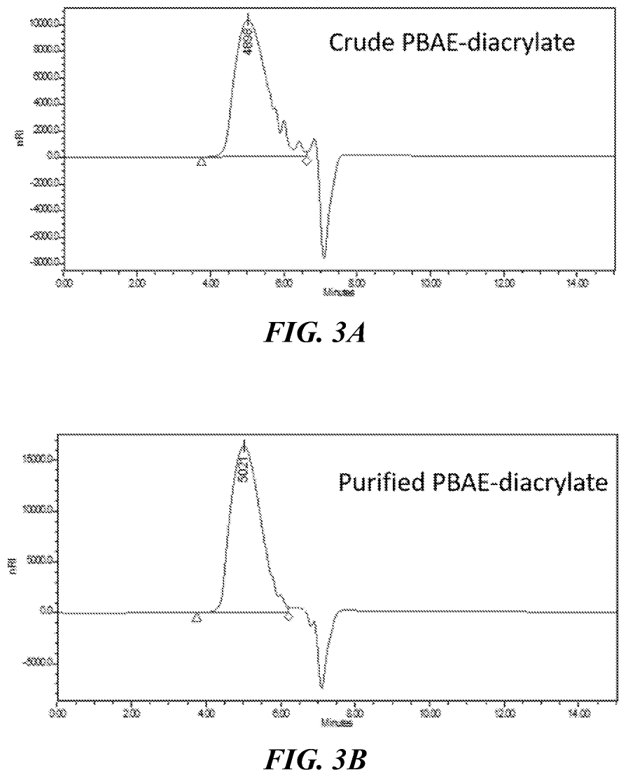

Therefore, a part of the synthesized PBAE-diacrylate polymers was purified by precipitation in Heptane. Crude product was dissolved in ethyl acetate and added dropwise into excess heptane (1/10, v/v), this procedure being repeated twice; or polymer was dissolved in ethyl acetate and heptane was added gradually to precipitate the polymer at a ratio of heptane to ethyl acetate of 2/1 (v/v). Purified PBAE-diacrylate polymers were monitored by TLC, following KMnO₄ staining. TLC plates demonstrated that the most of the apolar impurities were removed after purification by precipitation (FIG. 2, lanes 2 and 3). Moreover, purification by ethyl acetate/heptane at a ratio of 1/2 (v/v) seemed to impact the molecular weight characteristics greatly, degree of polymerization (DP) obtained by NMR for crude product was 7, DP of PBAE-diacrylate purified by ethyl acetate/heptane at 1/2 ratio was 17 and DP for PBAE-diacrylate purified by ethyl acetate/heptane at 1/10 ratio was 10. Therefore, ethyl acetate/heptane at the ratio of 1/10 (v/v) was selected as solvent system for the purification of PBAE-diacrylate. Purified PBAE-diacrylate using ethyl acetate/heptane (1/10, v/v) was obtained with an 86% yield and characterized by GPC to have M_{w}, and Mₙ, 5200 g/mol and 3300 g/mol, respectively. Moreover, GPC curves for crude PBAE-diacrylate obtained by classical method and purified PBAE-diacrylate polymers demonstrated that the small peaks at the low molecular weight region on the GPC trace of the crude product disappeared after the purification and the peak molecular weight moved slightly to a higher value (4900 and 5000, for crude and purified polymer, respectively) (compare FIG. 3A and FIG. 3B).

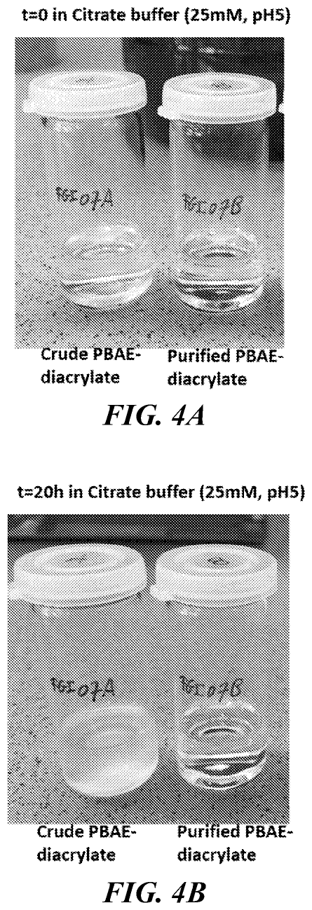

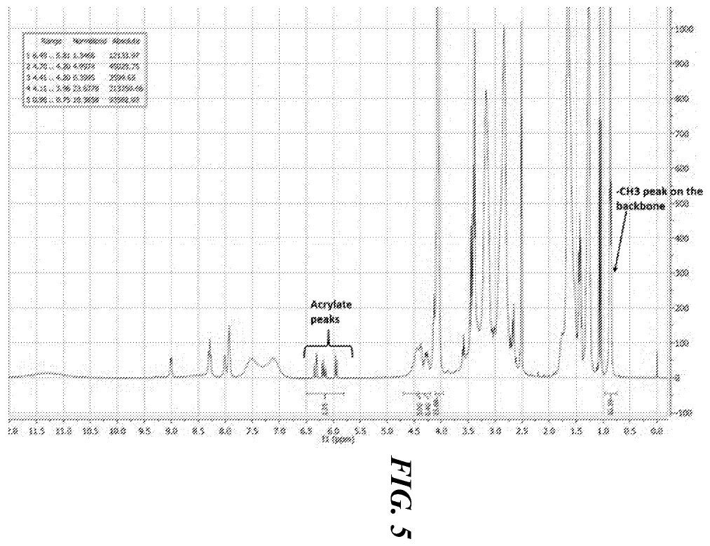

Furthermore, a physical appearance test was performed to determine the stability of crude vs purified PBAE-diacrylate polymers in 25 mM citrate buffer at pH 5.0 which is the solvent system used in functional cell assays. Both polymers were dissolved in citrate buffer and stirred at 25° C. for 20 h. At t=0, both polymers were soluble, resulting in clear-transparent solutions. However, at t=20 h, the solution of crude PBAE-diacrylate became cloudy. On the other hand, at t=20 h, the solution of purified polymer was still clear (see FIG. 4A and FIG. 4B).

Synthesis of OM-PBAE in DMSO using Purified PBAE-diacrylates as Starting Material

OM-PBAE polymers were obtained by peptide end-modification of PBAE-diacrylate polymers via thiol-acrylate Michael addition reaction in DMSO at a thiol/diacrylate ratio of 2.8:1. Synthesis of tri-arginine modified PBAE polymer (PBAE-CR3) is given as an example: purified PBAE-diacrylate polymer (199 mg, 0.08 mmol) was dissolved in DMSO (1.1 mL) and a hydrochloride salt of NH₂-Cys-Arg-Arg-Arg-COOH peptide (SEQ ID NO:4) (CR3—95% purity—purchased from Ontores) (168 mg, 0.23 mmol) was dissolved in DMSO (1 mL). Then the solutions of polymer and peptide were mixed and stirred at 25° C. in a temperature-controlled water bath for 20 h. Peptide modified PBAE was precipitated in 20 mL diethylether/acetone (7/3, v/v), then the product was washed two times with 7.5 mL diethylether/acetone (7/3, v/v), followed by vacuum drying and resulting product was resuspended in DMSO at a concentration of 100 mg/mL and stored at −20° C. for further use.

In a further example, tri-lysine end-modified PBAE polymer was obtained by mixing a solution of purified PBAE-diacrylate (199 mg, 0.08 mmol) dissolved in DMSO (1.1 mL) and a solution of hydrochloride salt of NH₂-Cys-Lys-Lys-Lys-COOH (SEQ ID NO:7) (CK3) (149 mg, 0.23 mmol) in DMSO (1 mL) and purification step was followed as previously described for PBAE-CR3. For the tri-histidine end-modified PBAE polymer, PBAE-CH3, a solution of purified PBAE-diacrylate (199 mg, 0.08 mmol) was dissolved in DMSO (1.1 mL) and mixed with a solution of hydrochloride salt of NH₂-Cys-His-His-His-COOH (SEQ ID NO:1) (CH3) (154 mg, 0.23 mmol) in DMSO (1.0 mL). For the tri-aspartate end-modified PBAE polymer, PBAE-CD3, a solution of purified PBAE-diacrylate (199 mg, 0.08 mmol) in DMSO (1.1 mL) was mixed with a solution of hydrochloride salt of NH₂-Cys-Asp-Asp-Asp-COOH (SEQ ID NO:13) (CD3) (114 mg, 0.23 mmol) in DMSO (1 mL). Finally, for the tri-glutamate end-modified PBAE polymer, PBAE-CE3, the solution of PBAE-diacrylate (199 mg, 0.08 mmol) in DMSO (1.1 mL) was mixed with a solution of hydrochloride salt of NH₂-Cys-Glu-Glu-Glu-COOH (SEQ ID NO:10) (CE3) (124 mg, 0.23 mmol) in DMSO (1 mL).

1H-NMR analysis confirmed the expected structures. Further, the percentage of acrylate conversion was determined from the ratio of CH₃ protons (0.8 ppm) on the polymer backbone which was calibrated to the same value as in the spectrum of the starting material to residual acrylate peaks (5.75-6.5 ppm). Therefore, dividing the integration value of acrylate peaks to six (which is the number of protons on the acrylate groups) yielded the residual acrylate amount. Michael addition reaction efficiencies for peptide modified PBAEs using purified PBAE-diacrylate were reported as; PBAE-CR3: 92%, PBAE-CK3: 98%, PBAE-CH3: 94%, PBAE-CD3: 96% and PBAE-CE3: >95%. Table 1 demonstrates that implemented purification step in the synthesis of PBAE-diacrylates did not have a negative influence on the Michael addition reaction efficiency; on the contrary, using purified backbone as a starting material even increased the acrylate conversion particularly for basic peptides (CK3, CH3 and CR3 (Table 1, entries 6, 7 and 8).

TABLE 1
Comparison of OM-PBAE synthesis efficiencies in DMSO via classical method and the new method
comprising a purification step for PBAE-diacrylates.

| | | | | Residual | |
|---|---|---|---|---|---|
| | | Crude/purified | Acrylate | peptide | |
| Entry | Peptide coupled | PBAE-diacrylate | conversion % | (% w/w) | Yield (wt %) |
| 1 | CK3 | Crude | 93% | n.d. | 131% |
| 2 | CH3 | Crude | 93% | 0.21 | 135% |
| 3 | CR3 | Crude | 84% | 1.12 | 158% |
| 4 | CD3 | Crude | 96% | <0.001 | 109% |
| 5 | CE3 | Crude | 98% | 0.31 | 101% |
| 6 | CK3 | Purified | 98% | n.d. | 149% |
| 7 | CH3 | Purified | 94% | 1.01 | 158% |
| 8 | CR3 | Purified | 92% | 2.95 | 166% |
| 9 | CD3 | Purified | 96% | 0.36 | 113% |
| 10 | CE3 | Purified | >95% | 1.55 | 105% |

Selection of New Solvent Systems for DMSO-free OM-PBAE Synthesis

Solubility testing was performed to determine the common solvents for PBAE-diacrylate, tetrapeptide hydrochloride salts and OM-PBAEs to further attempt a DMSO-free synthesis of peptide modified PBAEs. The tested compounds were dissolved in the different solvents at a concentration of 10-20 mg/mL and macroscopic aspect of the solutions was visually checked after incubation at 25° C. for 1 h. Three different solvent systems (methanol, ethanol and acetonitrile/citrate (25 mM, pH 5.0) (3/2, v/v) were selected based on the solubilities of PBAE-diacrylate, peptides and OM-PBAEs as shown in FIG. 1.

Stability of PBAE-Diacrylates in Methanol, Ethanol and Acetonitrile/Citrate (25 mM, pH 5.0) (3/2, v/v)

Initially, PBAE-diacrylate polymer was dissolved and incubated in methanol (100 mg/mL), ethanol (100 mg/mL) and acetonitrile/citrate (25 mM, pH 5.0) (3/2, v/v) (50 mg/mL) at 25° C. for 20 h. After removal of all residual solvents by rotary evaporator under reduced pressure, molecular weights of PBAE-diacrylate polymers were determined by a GPC system using a Waters HPLC system equipped with a GPC SHODEX KF-603 column (6.0×about 150 mm), and THF as mobile phase and with a refractive index (RI) detector. The molecular weights were calculated using a conventional calibration curve obtained by polystyrene standards. PBAE-diacrylate incubated in ethanol and acetonitrile/citrate (25 mM, pH 5.0) (3/2, v/v) did not show a significant change in molecular weights (before ethanol incubation: Mw=5600 g/mol, Mn=3400 g/mol; after incubation in ethanol: Mw=5700 g/mol, Mn=3400 g/mol; before acetonitrile/citrate incubation: Mw=7000 g/mol, Mn=3800 g/mol; after incubation in acetonitrile/citrate: Mw=6300 g/mol, Mn=3400 g/mol). On the other hand, incubation in methanol significantly reduced the molecular weight (before methanol incubation: Mw=5200 g/mol, Mn=3300 g/mol; after incubation in methanol: Mw=2700 g/mol, Mn=1800 g/mol). Therefore, methanol was not selected as a possible reaction solvent.

DMSO-Free Synthesis of OM-PBAE in Ethanol

OM-PBAE polymers were prepared by peptide end-modification of PBAE-diacrylate polymers via thiol-acrylate Michael addition reaction in ethanol at a thiol/diacrylate ratio of 2.8:1. Synthesis of tri-arginine modified PBAE polymer (PBAE-CR3) is given as an example: crude PBAE-diacrylate polymer (199 mg, 0.08 mmol) was dissolved in ethanol (2 mL) and a hydrochloride salt of NH₂-Cys-Arg-Arg-Arg-COOH peptide (SEQ ID NO:4) (CR3—95% purity—purchased from Ontores) (168 mg, 0.23 mmol) was dissolved in ethanol (12 mL). Then the solutions of polymer and peptide were mixed which led to the formation of a suspension. The resulting suspension stirred for 1 day at 25° C., then the reaction mixture was diluted with 11 mL of additional ethanol (final volume was 25 mL) and centrifuged. The resulting pellet was extracted twice with 7.5 mL ethanol and ethanol extracts were dried and analyzed by 1H-NMR to calculate acrylate conversion efficiency from the ratio of CH₃ protons (0.8 ppm) on the polymer backbone to residual acrylate peaks (5.75-6.5 ppm). The same procedure followed for CK3, CH3 and CD3 peptides. For all the peptides tested, the Michael addition reaction efficiency was less than 30%, so ethanol was demonstrated as a reaction solvent with low reaction rates.

DMSO-Free Synthesis of OM-PBAEs in Acetonitrile/Citrate (25 mM, pH 5.0) (3/2, v/v) with a Post-Coupling Purification Step

OM-PBAE polymers were obtained by peptide end-modification of PBAE-diacrylate polymers via thiol-acrylate Michael addition reaction in acetonitrile/citrate (25 mM, pH 5.0) (3/2, v/v) at a thiol/diacrylate ratio of 2.8:1. Crude PBAE-diacrylates were used. Synthesis of tri-arginine modified PBAE polymer (PBAE-CR3) is given as an example: crude PBAE-diacrylate polymer (199 mg, 0.08 mmol) was dissolved in acetonitrile (2 mL) and a hydrochloride salt of NH₂-Cys-Arg-Arg-Arg-COOH peptide (SEQ ID NO:4) (CR3—95% purity—purchased from Ontores) (168 mg, 0.23 mmol) was dissolved in 25 mM citrate buffer at pH 5.0 (4 mL). After complete dissolution of peptide, 4 mL acetonitrile was added to peptide solution. Then the solution of polymer was added to the peptide solution and stirred at 25° C. in a temperature-controlled water bath for 20 h. Then, all the solvents were evaporated at 40° C. under reduced pressure. As an additional improvement to the classical protocol, an extraction step was added to remove unreacted tetrapeptide salts and other side products from the final product. Resulting pellet was extracted with 10 mL of ethanol, twice. Ethanol extracts were dried. The resulting product was redissolved in 5 mL of ethanol and precipitated in 20 mL diethylether/acetone (7/3, v/v), then product was washed two times with 7.5 mL diethylether/acetone (7/3, v/v), followed by vacuum drying and stored at −20° C. for further use.

In a further example, tri-lysine end-modified PBAE polymer was obtained by mixing a solution of crude PBAE-diacrylate (199 mg, 0.08 mmol) dissolved in acetonitrile (2 mL) and a hydrochloride salt of NH2-Cys-Lys-Lys-Lys-COOH(CK3) (SEQ ID NO:7) (149 mg, 0.23 mmol) was dissolved in 25 mM citrate buffer at pH 5.0 (4 mL). After complete dissolution of peptide, 4 mL acetonitrile was added to peptide solution. Then, the polymer solution in acetonitrile was added to the peptide solution in acetonitrile/citrate (25 mM, pH 5.0) (1/1, v/v) and stirred 20 h at room temperature. Purification step was followed as previously described for PBAE-CR3. For the tri-histidine end-modified PBAE polymer, PBAE-CH3, the solution of PBAE-diacrylate (199 mg, 0.08 mmol) dissolved in acetonitrile (2 mL) that was mixed with a solution of hydrochloride salt of NH₂-Cys-His-His-His-COOH (SEQ ID NO:1) (CH3) (154 mg, 0.23 mmol) in acetonitrile/citrate (25 mM, pH 5.0) (1/1, v/v) (8 mL). The tri-aspartate end-modified PBAE polymer, PBAE-CD3, the solution of PBAE-diacrylate (199 mg, 0.08 mmol) dissolved in acetonitrile (2 mL) that was mixed with a solution of hydrochloride salt of NH₂-Cys-Asp-Asp-Asp-COOH (SEQ ID NO:13) (CD3) (114 mg, 0.23 mmol) in acetonitrile/citrate (25 mM, pH 5.0) (1/1, v/v) (8 mL). Finally, the tri-glutamate end-modified PBAE polymer, PBAE-CE3, the solution of PBAE-diacrylate (199 mg, 0.08 mmol) dissolved in acetonitrile (2 mL) that was mixed with a solution of hydrochloride NH₂-Cys-Glu-Glu-Glu-COOH (SEQ ID NO:13) (CE3) (124 mg, 0.23 mmol) in acetonitrile/citrate (25 mM, pH 5.0) (1/1, v/v) (8 mL). For PBAE-CD3 and PBAE-CE3, a post-reaction purification protocol which was slightly different from the one previously described for PBAE-CR3, PBAE-CH3 and PBAE-CK3 was applied due to the different solubility properties of PBAE-CD3 and PBAE-CE3. Resulting polymers were dissolved in water (1 mL) and precipitated in ethanol (10 mL) twice, further dried under vacuum and stored in solid form for further use at −20° C.

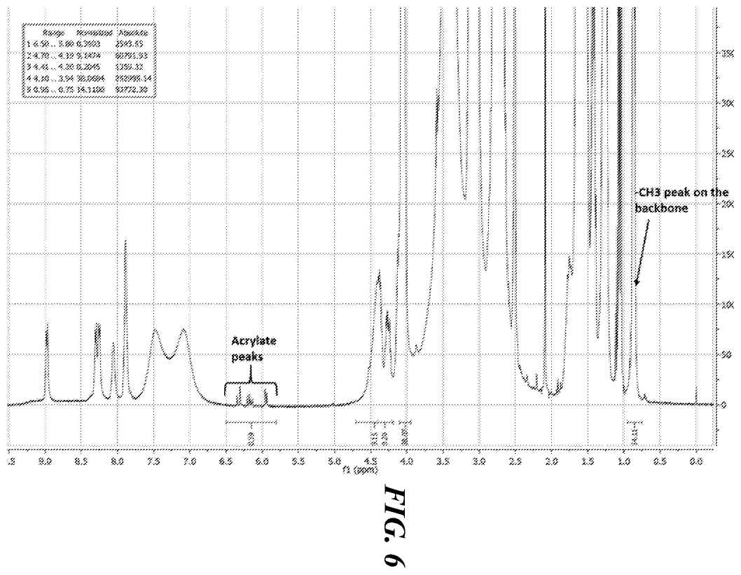

1H-NMR analysis was used to confirm the expected structures. Moreover, the percentage of acrylate conversion was determined from the ratio of CH₃ protons (0.8 ppm) on the polymer backbone to residual acrylate peaks (5.75-6.5 ppm) (FIG. 5, a representative NMR spectrum of PBAE-CR3). The acrylate conversions for peptide modified PBAEs from crude PBAE-diacrylate were determined as; PBAE-CR3: 78%, PBAE-CK3: 93%, PBAE-CH3: 86%, PBAE-CD3: 100% and PBAE-CE3: 100%. Recapitulative results of OM-PBAE synthesis in DMSO and in DMSO-free conditions are shown in Table 2.

The residual peptide content in each peptide modified PBAE was quantified by UV detection (wavelength 220 nm) after separation by UPLC ACQUITY system (Waters) equipped with a BEH C18 column (130 A, 1.7 μm, 2.1×50 mm, temperature 35° C.) using an acetonitrile/water with 0.1% TFA as gradient.

TABLE 2
Comparison of DMSO and new DMSO free synthesis of OM-PBAEs using the same starting
materials (crude PBAE-diacrylate polymers were used)

| | | | | Residual | |
|---|---|---|---|---|---|
| | | | Acrylate | peptide | |
| Entry | Peptide coupled | Reaction solvent | conversion % | (% w/w) | Yield (wt %) |
| 1 | CK3 | DMSO | 93% | n.d. | 131% |
| 2 | CH3 | DMSO | 93% | 0.21 | 135% |
| 3 | CR3 | DMSO | 84% | 1.12 | 158% |
| 4 | CD3 | DMSO | 96% | <0.001 | 109% |
| 5 | CE3 | DMSO | 98% | 0.31 | 101% |
| 11 | CK3 | Acetonitrile/citrate | 93% | n.d. | 29% |
| 12 | CH3 | Acetonitrile/citrate | 86% | n.d. | 32% |
| 13 | CR3 | Acetonitrile/citrate | 78% | 0.02 | 44% |
| 14 | CD3 | Acetonitrile/citrate | 100% | 13.45 | 56% |
| 15 | CE3 | Acetonitrile/citrate | 100% | 3.53 | 62% |

DMSO-Free Synthesis of OM-PBAEs in Acetonitrile/Citrate (25 mM, pH 5.0) (3/2, v/v) with Purified PBAE-Diacrylate and Including a Post-Reaction Purification Step

OM-PBAE polymers were obtained by peptide end-modification of PBAE-diacrylate polymers via thiol-acrylate Michael addition reaction in acetonitrile/citrate (25 mM, pH 5.0) (3/2, v/v) at a thiol/diacrylate ratio of 2.8:1. Purified PBAE-diacrylates were used. Synthesis of tri-arginine modified PBAE polymer (PBAE-CR3) is given as an example: purified PBAE-diacrylate polymer (199 mg, 0.08 mmol) was dissolved in acetonitrile (2 mL) and a hydrochloride salt of NH₂-Cys-Arg-Arg-Arg-COOH peptide (SEQ ID NO:4) (CR3—95% purity—purchased from Ontores) (168 mg, 0.23 mmol) was dissolved in 25 mM citrate buffer at pH 5.0. After complete dissolution of peptides, 4 mL acetonitrile was added to the peptide solution. Then the solution of polymer was added to the peptide solution and stirred at 25° C. in a temperature-controlled water bath for 20 h. Then, all the solvents were evaporated at 40° C. under reduced pressure. Resulting pellet was extracted with 10 mL of ethanol, twice. Ethanol extracts were dried. The resulting product was redissolved in 5 mL of ethanol and precipitated in 20 mL diethylether/acetone (7/3, v/v), then product was washed two times with 7.5 mL diethylether/acetone (7/3, v/v), followed by vacuum drying and stored at −20° C. for further use.

In a further example, tri-histidine end-modified PBAE polymer, PBAE-CH3 the solution of PBAE-diacrylate (199 mg, 0.08 mmol) dissolved in acetonitrile (2 mL) that was mixed with a solution of hydrochloride salt of NH₂-Cys-His-His-His-COOH (SEQ ID NO:1) (CH3) (154 mg, 0.23 mmol) in Acetonitrile/citrate (25 mM, pH 5.0) (1/1, v/v) (8 mL). A similar purification protocol is followed as described for PBAE-CR3.

1H-NMR was used to determine the Michael addition reaction efficiency in acetonitrile/citrate (25 mM, pH 5.0) (3/2, v/v). The percentage of acrylate conversion was determined from the ratio of CH₃ protons (0.8 ppm) on the polymer backbone to residual acrylate peaks (5.75-6.5 ppm). The acrylate conversion ratios for peptide modified PBAEs from purified PBAE-diacrylate were determined as PBAE-CR3: 85% and PBAE-CH3: 95%.

DMSO-Free Synthesis of OM-PBAEs in Acetonitrile/Citrate (25 mM, pH 5.0) (3/2, v/v) with Purified PBAE-Diacrylate and Using Two-Times Concentrated Peptide Solution and Including a Post-Coupling Purification Step

Additional developments of the synthesis consisted in increasing the efficiency of the acrylate conversion during the CRRR peptide end-modification step and finding extraction/purification conditions that would separate uncoupled PBAE-diacrylate byproducts from fully converted OM-PBAEs. OM-PBAE polymers were obtained by peptide end-modification of PBAE-diacrylate polymers via thiol-acrylate Michael addition reaction in acetonitrile/citrate (25 mM, pH 5.0) (3/2, v/v) at a thiol/diacrylate ratio of 2.8:1. Purified PBAE-diacrylates and two times concentrated peptide solution were used to reduce the reaction volume and favor the reaction towards the conversion of acrylates. Synthesis of tri-arginine modified PBAE polymer (PBAE-CR3) is given as an example: purified PBAE-diacrylate polymer (199 mg, 0.08 mmol) was dissolved in acetonitrile (1 mL) and a hydrochloride salt of NH₂-Cys-Arg-Arg-Arg-COOH peptide (SEQ ID NO:4) (CR3—95% purity—purchased from Ontores) (168 mg, 0.23 mmol) in 25 mM citrate buffer at pH 5.0. After complete dissolution of peptides, 4 mL acetonitrile was added to the peptide solution. Then the solution of polymer was added to the peptide solution and stirred at 25° C. in a temperature-controlled water bath for 20 h. Then, all the solvents were evaporated at 40° C. under reduced pressure. Resulting pellet was extracted with 10 mL of ethanol, twice. Ethanol extracts were dried. The dry rest was redissolved in 5 mL of ethanol and precipitated in 20 mL diethylether/acetone (7/3, v/v), then product was washed two times with 7.5 mL diethylether/acetone (7/3, v/v), followed by vacuum drying and stored at −20° C. for further use (Entry 18). Alternatively, pellet was extracted three times with 15 mL of ethanol, ethanol extracts were combined and dried, and dry rest was precipitated in 20 mL diethylether/acetone (7/3, v/v) after dissolving 5 mL ethanol (Entry 19); ii) pellet was extracted twice with 10 mL ethanol, ethanol extracts were combined and dried, and dry rest was precipitated in 20 mL diethylether/acetone (7/3, v/v) after dissolving 5 mL ethanol/water (4/1, v/v) (Entry 20); or iii) 5 mL ethanol/water (3/2, v/v) (Entry 21).

1H-NMR was used to confirm the structure and to determine the Michael addition reaction efficiency in acetonitrile/citrate (25 mM, pH 5.0) (3/2, v/v). The percentage of acrylate conversion was determined from the ratio of CH₃ protons (0.8 ppm) on the polymer backbone to residual acrylate peaks (5.75-6.5 ppm) (FIG. 6). The acrylate conversion for peptide modified PBAEs from purified PBAE-diacrylate and concentrated reaction mixture was determined as; PBAE-CR3: >90%. Moreover, a summary of DMSO-free synthesis and purification of PBAE-CR3 and PBAE-CH3 under different conditions is given in Table 3. If acrylate conversion was improved over 90% by using a two times concentrated peptide solution, the more extensive purification steps resulted in lower overall reaction yields.

TABLE 3
Summary of different reaction and purification conditions for the synthesis of PBAE-CR3 and PBAE-CH3
in DMSO-free conditions.

| | Peptide | | Acrylate | Yield |
|---|---|---|---|---|
| Entry | coupled | Condition | conversion % | (wt %) |
| 16 | CR3 | crude backbone, 2× extracted in EtOH, | 78% | 39% |
| | | Dissolved in EtOH & 1× precipitated in diethylether/acetone | | |
| 17 | CR3 | purified backbone, 2× extracted in EtOH, | 85% | 44% |
| | | Dissolved in EtOH & 1× precipitated in diethylether/acetone | | |
| 18 | CR3 | purified backbone, 2× concentrated peptide sln, 2× extracted | 90% | 44% |
| | | in EtOH, Dissolved in EtOH & 1× precipitated in | | |
| | | diethylether/acetone | | |
| 19 | CR3 | purified backbone, 2× concentrated peptide sln, 3× extracted | 92% | 35% |
| | | in EtOH, Dissolved in EtOH & 1× precipitated in | | |
| | | diethylether/acetone | | |
| 20 | CR3 | purified backbone, 2× concentrated peptide sln, 2× extracted | 90% | 24% |
| | | in EtOH, dissolved in EtOH/H2O (4/1) & 1× precipitated in | | |
| | | diethylether/acetone | | |
| 21 | CR3 | purified backbone, 2× concentrated peptide sln, 2× extracted | 94% | 16% |
| | | in EtOH, dissolved in EtOH/H2O (3/2) & 1× precipitated in | | |
| | | diethylether/acetone | | |
| 22 | CH3 | crude backbone, 2× extracted in EtOH, | 86% | 32% |
| | | Dissolved in EtOH & 1× precipitated in diethylether/acetone | | |
| 23 | CH3 | purified backbone, 2× extracted in EtOH, Dissolved in EtOH | 95% | 58% |
| | | & 1× precipitated in diethylether/acetone | | |

DMSO-Free 1 g Scale Synthesis of OM-PBAE

1 g scale synthesis of OM-PBAEs was performed in acetonitrile/citrate (25 mM, pH 5.0) (3/2, v/v) using purified PBAE-diacrylate precursor polymer and 2× concentrated peptide solution as described in Table 3, entry 18. In addition to protocol described for entry 18, inert nitrogen (N₂) atmosphere was applied during the reaction to prevent di-sulfide formation in the reaction medium. Synthesis of tri-arginine modified PBAE polymer (PBAE-CR3) was given as an example. Purified PBAE-diacrylate polymer (1999 mg, 0.624 mmol) was dissolved in acetonitrile (20 ml) and a hydrochloride salt of NH₂-Cys-Arg-Arg-Arg-COOH peptide (SEQ ID NO:4) (CR3—97% purity—purchased from Ontores) (1684 mg, 2.3 mmol) was dissolved in citrate buffer (25 mM, pH 5.0) (20 ml), after complete dissolution of peptide 10 ml acetonitrile was added. Then the solution of polymer in acetonitrile was added to the peptide solution in citrate (25 mM, pH 5.0)/acetonitrile (2/1, v/v) and stirred at 25° C. in a temperature-controlled water bath for 20 h under N₂ atmosphere. Then, all the solvents were evaporated at 40° C. under reduced pressure. Resulting pellet was extracted with 100 ml of ethanol, twice. Ethanol extracts were dried. The dry rest was re-dissolved in 50 ml of ethanol and precipitated in 200 ml diethylether/acetone (7/3, v/v), then product was washed two times with 75 ml diethylether/acetone (7/3, v/v). Residual organic solvents were removed under vacuum, further final product was obtained by lyophilization with a 37% (wt %) yield and stored at −20° C. for further use. The rest of the characteristics of the 1 g scale PBAE-CR3 are given in Table 4, entry 24.

For tri-histidine end modified PBAE polymer (PBAE-CH3), purified PBAE-diacrylate polymer (1999 mg, 0.624 mmol) was dissolved in acetonitrile (20 ml) and a hydrochloride salt of NH2-Cys-His-His-His-COOH peptide (CH3—98% purity—purchased from Ontores) (1.538 mg, 2.3 mmol) was dissolved in 20 ml 25 mM citrate buffer at pH 5.0. After complete dissolution of CH3 peptide 10 mL acetonitrile was added. Then the solution of polymer in acetonitrile was added to the peptide solution in acetonitrile/citrate (25 mM, pH 5.0) (2/1, v/v) and stirred at 25° C. in a temperature-controlled water bath for 20 h under inert N₂ atmosphere. Then, all the solvents were evaporated at 40° C. under reduced pressure. Resulting pellet was extracted with 100 ml of ethanol, twice. Ethanol extracts were dried. The dry rest was re-dissolved in 50 ml of ethanol and precipitated in 200 ml diethylether/acetone (7/3, v/v), then product was washed two times with 75 ml diethylether/acetone (7/3, v/v). Residual organic solvents were removed under vacuum, further final product was obtained with a 45.3% (wt %) yield and stored at −20° C. for further use. The rest of the characteristics of 1 g scale PBAE-CH3 are given in Table 4, entry 25.

Synthesis of tri-glutamic acid end modified PBAE polymer (PBAE-CE3) was carried out using the same procedure, i.e. purified PBAE-diacrylate polymer (3668 mg, 1.0 mmol) was dissolved in acetonitrile (24 ml) and a hydrochloride salt of NH₂-Cys-Glu-Glu-Glu-COOH peptide (SEQ ID NO:10) (CE3—97% purity—purchased from Ontores) (1516 mg, 2.78 mmol) was dissolved in 48 ml 25 mM citrate buffer at pH 5.0. After complete dissolution of peptide, 48 ml acetonitrile was added to peptide solution. Then, the solution of polymer in acetonitrile was added to the peptide solution in acetonitrile/citrate (25 mM, pH 5.0) (1/1, v/v) and stirred at 25° C. in a temperature-controlled water bath for 20 h under inert N2 atmosphere. Then, all the solvents were evaporated at 40° C. under reduced pressure. In order to achieve a better removal of unreacted peptides compared to small scale reactions performed with negatively charged oligopeptides (entries 14 and 15), the purification of PBAE-CE3 was modified to apply the reaction product onto a Sephadex PD Miditrap G-10 size exclusion columns using water at pH 7.2 as eluent. Then, collected polymer fractions were combined, lyophilized, and stored at −20° C. for further use. The specifications of CE3-PBAE was given in Table 5, entry 26.

TABLE 4
Characteristics of OM-PBAEs produced 1 g scale in DMSO-free
conditions.

| Entry | 24 | 25 | 26 |
|---|---|---|---|
| Peptide | CR3 | CH3 | CE3 |
| Acrylate conversion (%) | 99.5 | 93.7 | 90.3 |
| Yield | 37.0% | 45.3% | 70% |
| m (CH₃) | 4.3 | 5.6 | 6.5 |
| (0.85 ppm) | | | |
| m + n (CH₂—O) | 9.7 | 13.0 | 14 |
| (4.05 ppm) | | | |
| Mn from NMR (Da) | 4600 | 5500 | 5300 |
| Residual peptide content (% m/m) | 0.8 | 0.1 | 0.2 |

### Example 3. Use of OM-PBAEs for the Production of Polymer-Coated Lentiviral Vectors

Functional properties of OM-PBAEs synthetized according to the different above-described protocols were evaluated based on their ability to form polymer-coated lentiviral vectors for use in cell transduction studies. Polymer-coated lentiviral vectors were made using the following materials and methods.

Materials

The transfer vector plasmid was pARA-CMV-GFP with the gene encoding Green Fluorescent Protein (GFP). A kanamycin-resistant plasmid encoded for the provirus (a non-pathogenic and non-replicative recombinant proviral DNA derived from HIV-1, strain NL4-3), in which an expression cassette was cloned. The insert contained the transgene, the promoter for transgene expression and sequences added to increase the transgene expression and to allow the lentiviral vector to transduce all cell types including non-mitotic ones. The promoter was the human ubiquitin promoter or the CMV promoter. It was devoid of any enhancer sequence and it promoted gene expression at a high level in a ubiquitous manner. The non-coding sequences and expression signals corresponded to Long Terminal Repeat sequences (LTR) with the whole cis-active elements for the 5′LTR (U3-R-U5) and the deleted one for the 3′LTR, hence lacking the promoter region (AU3-R-U5). For the transcription and integration experiments, encapsidation sequences (SD and 5′Gag), the central PolyPurine Tract/Central Termination Site for the nuclear translocation of the vectors, and the BGH polyadenylation site were added.

The packaging plasmid was pARA-Pack. The kanamycin resistant plasmid encoded for the structural lentiviral proteins (GAG, POL, TAT and REV) used in trans for the encapsidation of the lentiviral provirus. The coding sequences corresponded to a polycistronic gene gag-pol-tat-rev, coding for the structural (Matrix MA, Capsid CA and Nucleocapsid NC), enzymatic (Protease PR, Integrase IN and Reverse Transcriptase RT) and regulatory (TAT and REV) proteins. The non-coding sequences and expression signals corresponded to a minimal promoter from CMV for transcription initiation, a polyadenylation signal from the insulin gene for transcription termination, and an HIV-1 Rev Responsive Element (RRE) participating for the nuclear export of the packaging RNA.

Production of VSV-G⁻ (“Bald”) Lentiviral Vector Particles

LV293 cells were seeded at 5×10⁵ cells/mL in 2×3000 mL Erlenmeyer flasks (Corning) in 1000 mL of LVmax Production Medium (Gibco Invitrogen). The two Erlenmeyers were incubated at 37° C., 65 rpm under humidified 8% CO₂. The day after seeding, the transient transfection was performed. PEIPro transfectant reagent (PolyPlus Transfection, Illkirch, France) was mixed with transfer vector plasmid pARA-CMV-GFP and packaging plasmid (pARA-Pack). After incubation at room temperature, the mix PEIPro/Plasmid was added dropwise to the cell line and incubated at 37° C., 65 rpm under humidified 8% CO₂. At day 3, the lentivector production was stimulated by sodium butyrate at 5 mM final concentration. The bulk mixture was incubated at 37° C., 65 rpm under humidified 8% CO₂ for 24 hours. After clarification by deep filtration at 5 and 0.5 μm (Pall Corporation), the clarified bulk mixture was incubated 1 hour at room temperature for DNase treatment.

Lentivector purification was performed by chromatography on a Q mustang membrane (Pall Corporation) and eluted by NaCl gradient. Tangential flow filtration was performed on a 100 kDa HYDROSORT membrane (Sartorius), which allowed to reduce the volume and to formulate in specific buffer at pH 7, ensuring at least 2 years of stability. After sterile filtration at 0.22 μm (Millipore), the bulk drug product was filled in 2 mL glass vials with aliquots less than 1 ml, then labelled, frozen and stored at <−70° C.

The bald LV number was evaluated by physical titer quantification. The assay was performed by detection and quantitation of the lentivirus associated HIV-1 p24 core protein only (Cell Biolabs Inc.). A pre-treatment of the samples allows to distinguish the free p24 from destroyed Lentivectors. Physical titer, particle distribution and size were measured by tunable resistive pulse sensor (TRPS) technology (qNano instrument, Izon Science, Oxford, UK). NP150 nanopore, 110 nm calibration beads and membrane stretch between 44 and 47 mm sere used. The results were determined using the IZON Control Suite software.

Coating of Bald Lentiviral Vectors with Oligopeptide-Modified PBAE

Coating of bald lentiviral vectors (8×10⁹ lentiviral viral particles) was performed with a ratio of 10⁹ polymer molecules per lentiviral vector particle as follows. Bald lentiviral vectors were diluted in 25 mM citrate buffer pH 5.4 to prepare a final volume of 75 μL per replicate. PBAE polymers were diluted in the same buffer as for lentiviral vectors (75 μL per replicate) and vortexed 2 s for homogenization. The diluted polymers were added to the diluted vectors in a 1:1 ratio (v/v), the mixes were gently vortexed for 10 s and incubated 10 minutes at room temperature. Finally, an equal volume of culture medium (150 μL) was added to the coated particles before transfer to cells.

### Example 4. Transduction of Human Lymphocytes by Polymer-Coated Lentiviral Vectors

OM-PBAEs have already been described as transfection agents that form polymer-encapsulated vehicles able to deliver genetic material (plasmids or other nucleic acid molecules) to eukaryotic cells (US2016/0145348A1, Mangraviti et al. 2015, Anderson et al. 2004, WO2016/116887). Here, OM-PBAEs were used to coat transduction-deficient lentiviral vectors and engineer human cells to stably express various transgenes including reporter genes (GFP and mCherry) and Chimeric Antigen Receptors (CARs) (WO2019145796).

In order to compare the properties of OM-PBAEs obtained with the different synthesis protocols, in vitro assays were carried out with polymer-coated lentiviral vectors encoding for a green fluorescent protein (GFP) and evaluated the impact of the changes in the synthesis and purification protocol on the transduction efficiency of human lymphocytes and on cell viability. The polymers used in the encapsulation experiments were poly(beta-amino esters) (PBAEs) conjugated to charged peptides. Polymer PBAE-CR3 refers to PBAE conjugated to the peptide CRRR (SEQ ID NO:4) (same peptide at both ends). PBAE-CH3 polymer refers to PBAE conjugated to the peptide CHHH (SEQ ID NO:1). Mixtures of these OM-PBAEs were tested as well at 60/40 and 40/60 v/v ratios.

Freshly prepared Peripheral Blood Mononuclear Cells (PBMCs) were isolated from buffy coats obtained from healthy donors (Etablissement Français du Sang, Division Rhônes-Alpes). After diluting the blood with DPBS, the PBMCs were separated over a FICOLL density gradient (GE Healthcare), washed twice with DPBS, resuspended to obtain the desired cell density and cultured in RPMI-1640 medium (Gibco Invitrogen), supplemented with 10% FBS (Gibco Invitrogen) and 1% Penicillin/Streptomycin (Gibco Invitrogen) at 37° C., 5% CO₂. In vitro assays were also performed with frozen Human lymphocytes preparations from healthy donors (Etablissement Français du Sang, Division Rhônes-Alpes) and cultured under the same conditions as PBMCs.

Human PBMCs and lymphocyte preparations were seeded in 24-well plates at a density of 10⁵ cells per well in RPMI medium containing 10% FBS and 1% penicillin/streptomycin. 300 μL of encapsulated vector were added to the cells. After 2 h incubation at 37° C., 5% CO₂, 500 μL of fresh complete medium was added to each well. The percentage of cells expressing GFP was determined 72 h post-transduction with an Attune NxT flow cytometer using the BL1 channel. The phenotype of transduced cells expressing GFP transgene was determined by flow cytometry staining with antibodies specific for the following cell types following manufacturer's instructions (BD Biosciences): CD3 (T lymphocytes) and CD19 (B lymphocytes). Cell viability was determined 72 h post-transduction with an Attune NxT flow cytometer (Thermo Fisher) by counting singlet alive cells in forward scatter—x side scatter-gated population excluding aggregates and cell debris. All conditions were tested as independent triplicates.

First, the impact was studied of PBAE-diacrylate purification on the biological properties of different mixes of PBAE-CH3 and PBAE-CR3 synthetized with the classical protocol (Entries 2 and 3) or submitted to a 1/10 EtOAc/heptane precipitation before peptide coupling (Entries 7 and 8). In such an experimental system “bald LV” (LV) are not capable of performing transduction due to the lack of VSV-G protein, but they become capable of transduction after their encapsulation by OM-PBAE polymers which gives rise to GFP-positive lymphocytes. Another control with untransduced cells (NT) is included to monitor background level of GFP expression, autofluorescence of culture medium components and cell viability during the experiment.

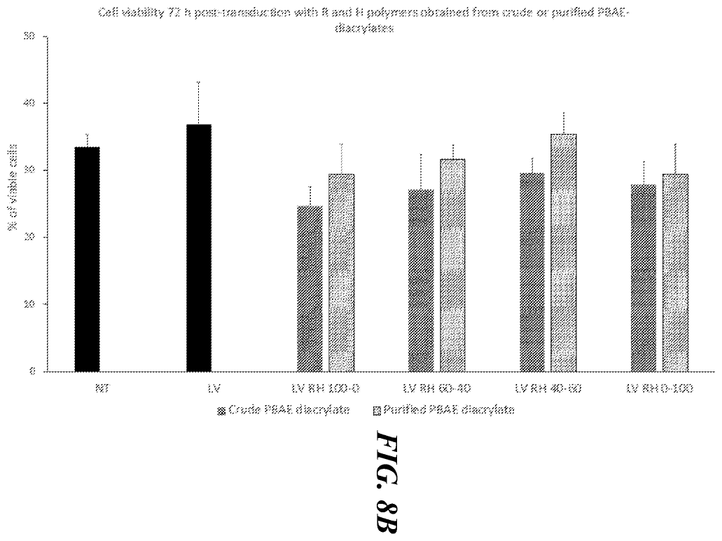

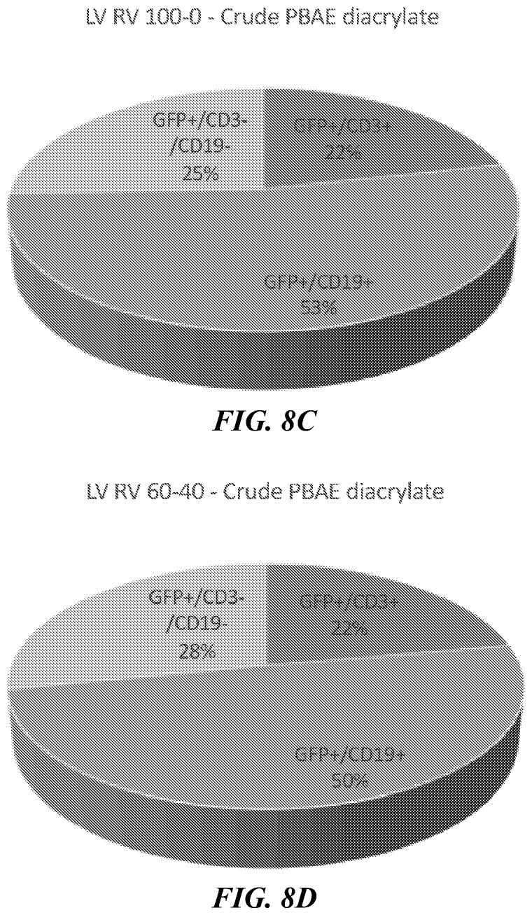

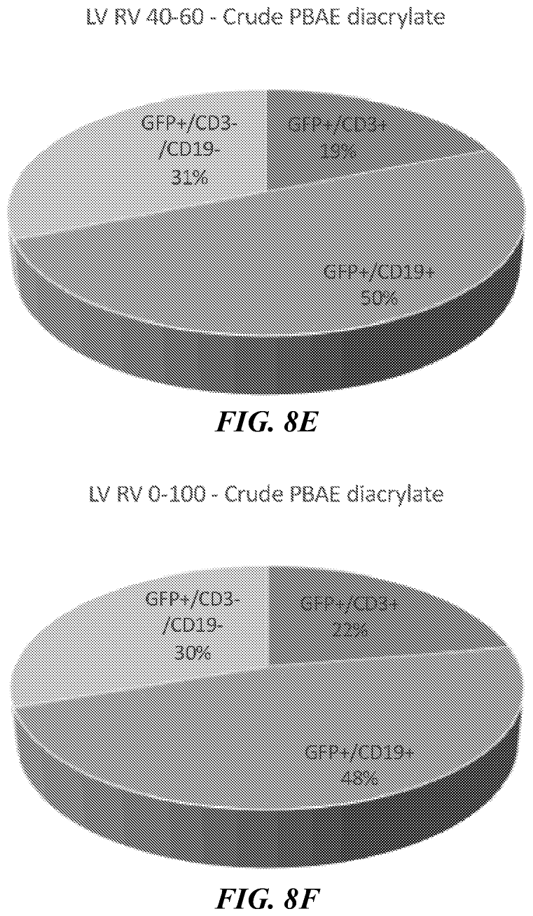

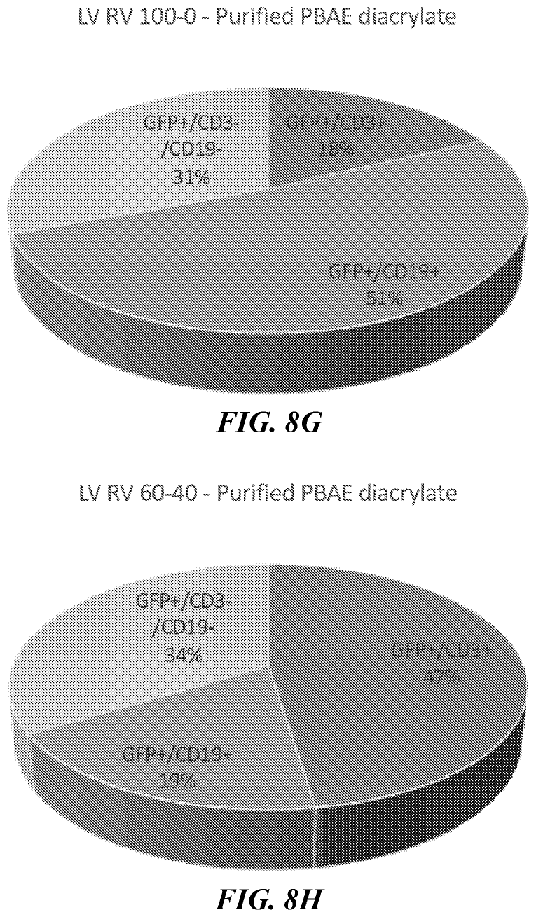

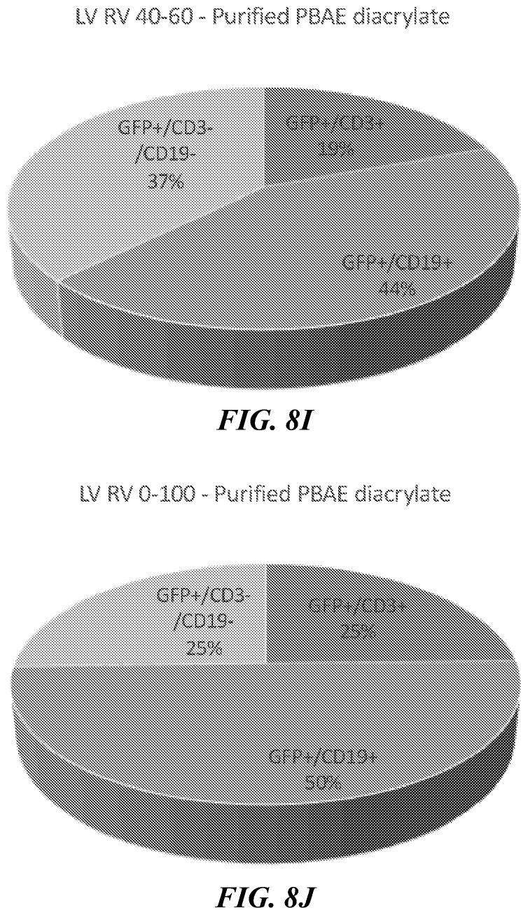

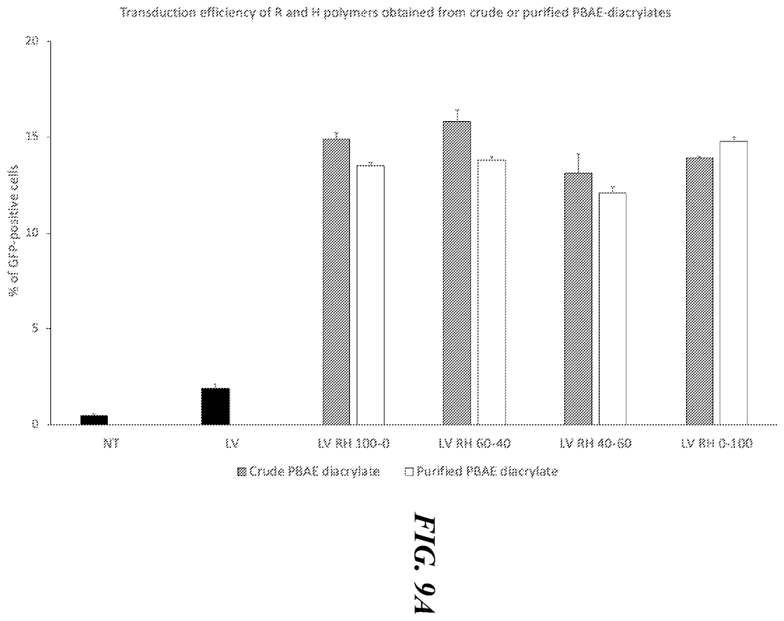

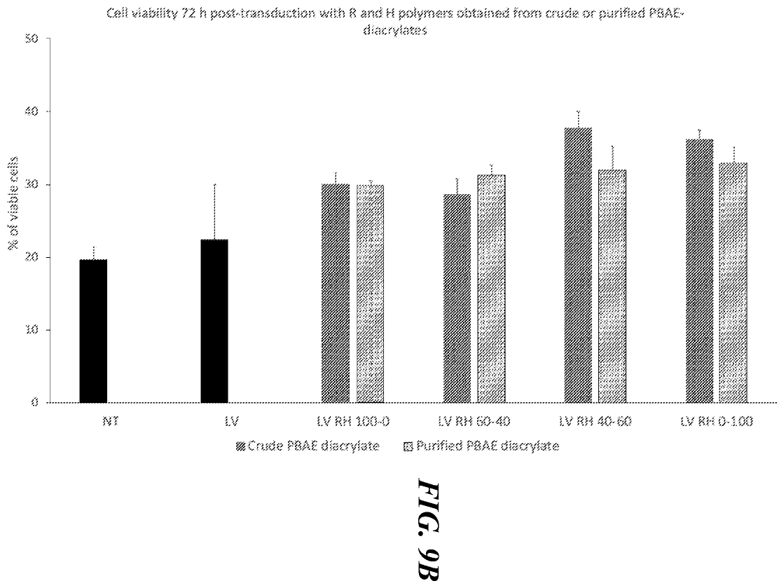

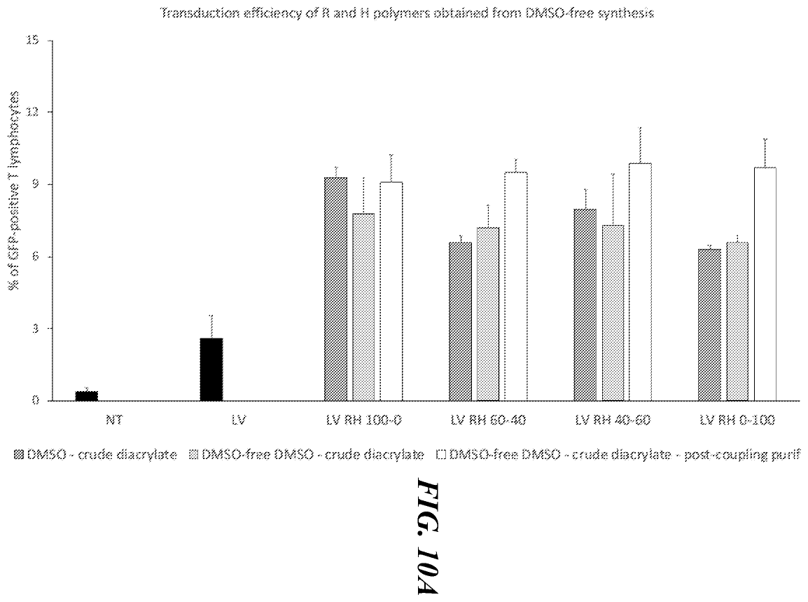

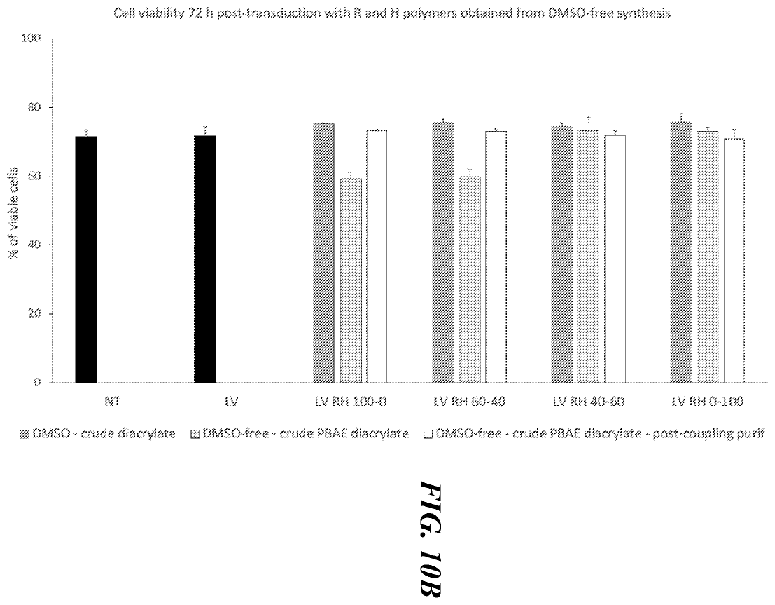

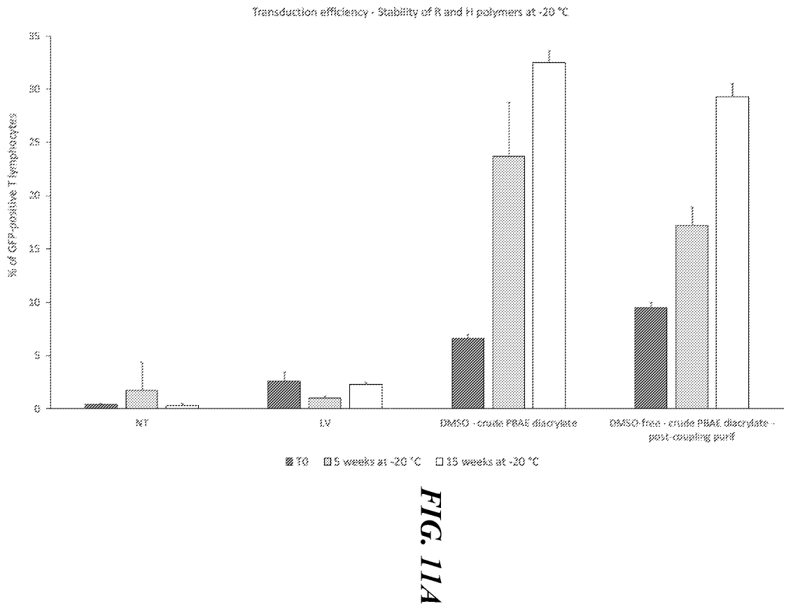

As shown in FIGS. 8A and 8B, no difference in transduction efficiency and cell toxicity was observed between polymers derived from PBAE purification or unprocessed ones as in the classical protocol. The PBAE-diacrylate purification did not modify the distribution of lymphocyte sub-populations transduced by polymer coated-lentiviral vectors (see FIGS. 8C-8J). The same results were obtained when cells from frozen Human lymphocyte preparation were replaced by freshly prepared PBMCs (FIGS. 9A and 9B).

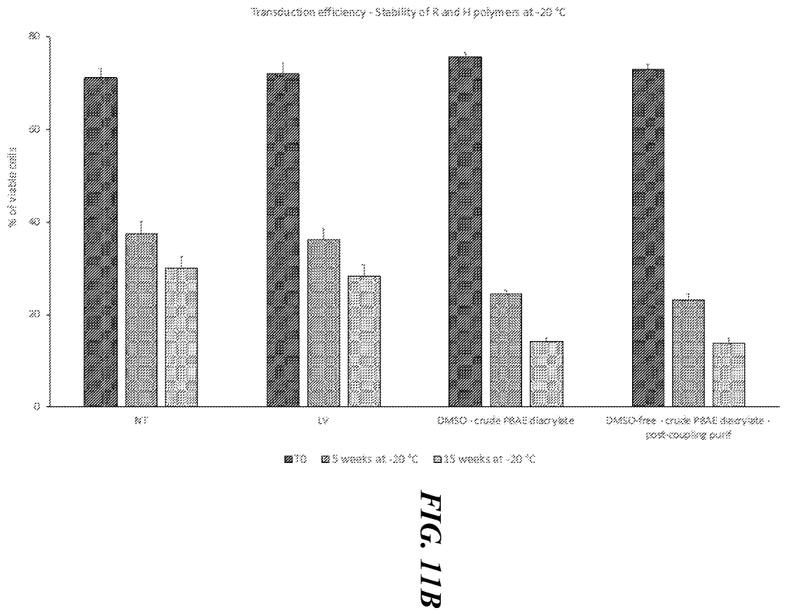

As next step, the impact of replacing DMSO by acetonitrile/citrate during the oligopeptide-end coupling reaction was studied, and again no difference in transduction efficiency and cell toxicity was observed when unprocessed reaction products were tested at different PBAE-CH3/PBAE-CR3 mixes (see FIGS. 10A and 10B). The introduction of a post-coupling purification step in the protocol slightly improved the transduction efficiencies.

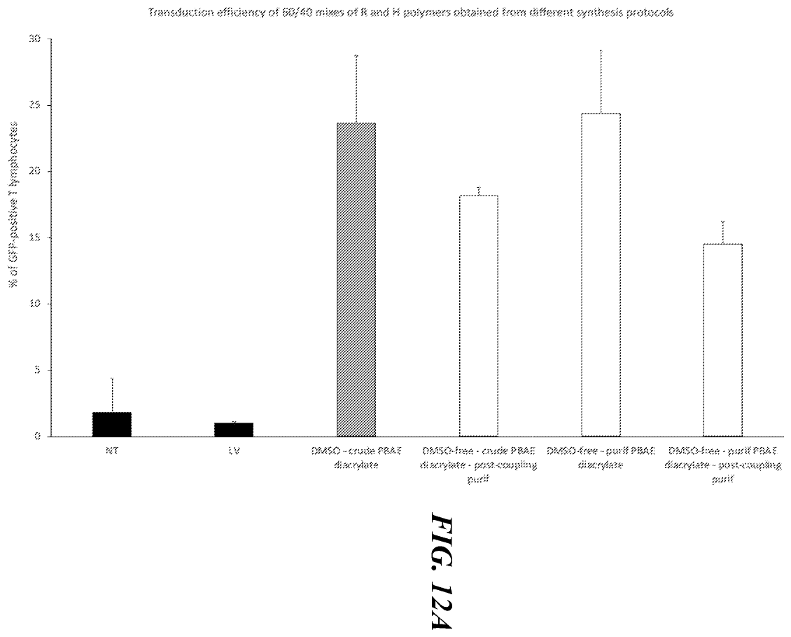

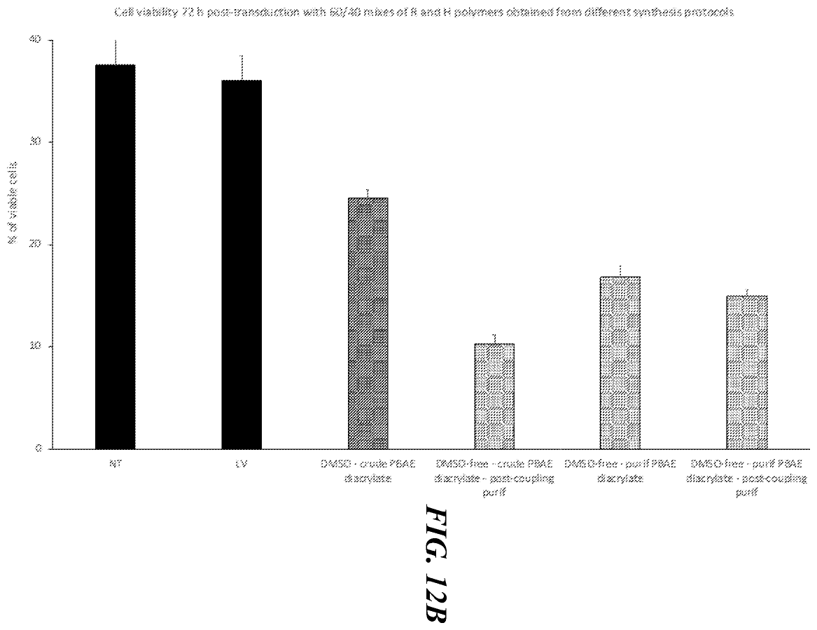

The DMSO-free oligopeptide coupling reaction produces lyophilized polymers with residual citrate that can be resuspended in buffers compatible with biological systems. Long-term stability is an issue with polymers of the PBAE family as they all have been reported to be degraded at pH 7.0 in aqueous buffers (Lynn et al. 2000). This stability issue has been circumvented in the past by storing PBAEs in DMSO at −20° C. Therefore, an investigation was performed of the functionality of 60/40 ratios of PBAE-CR3 and PBAE-CH3 obtained with DMSO-free coupling plus post-coupling purification (Entries 12 and 13) and freshly resuspended in water or stored in water for 5 or 15 weeks at −20° C. and compared their stability with polymers synthetized with the classical protocol (Entries 2 and 3) and stored in DMSO at −20° C. during the same period of time. Results summarized in FIGS. 11A and 11B clearly show polymers formulated in water are functional and that their transduction efficiency is not lost upon long-term storage in water. In three different PBMCs preparations DMSO-free OM-PBAEs were not more toxic to cells than those obtained with the classical protocol; which suggests that no degradation by-products are generated during the storage in aqueous conditions.

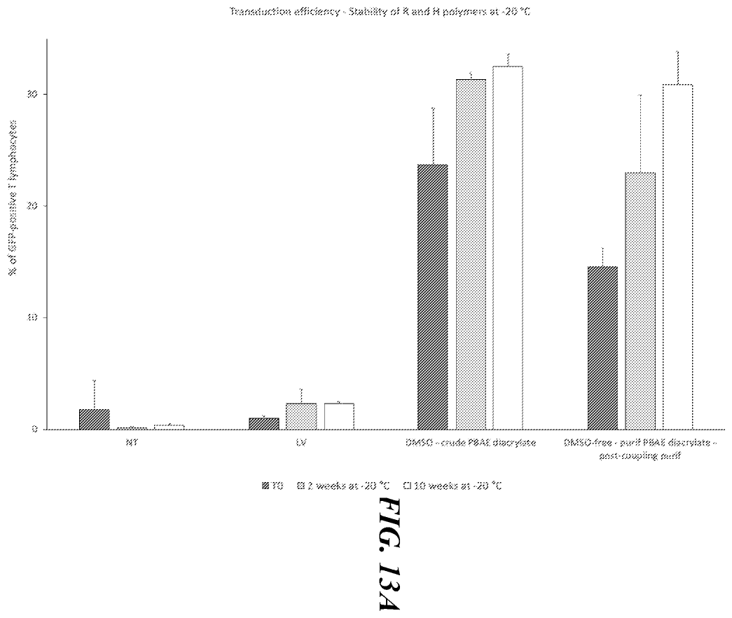

Next, the behavior was checked of OM-PBAEs obtained by the new synthesis protocol, which combines PBAE-diacrylate purification, DMSO-free oligopeptide coupling and post-coupling purification. If DMSO-free polymers generally appeared more toxic to PBMCs compared to those obtained with classical protocol, the addition of PBAE-diacrylate purification reduced observed toxicities (see FIG. 12B.) On the other hand, transduction efficiency was not impacted as shown in FIG. 12A.

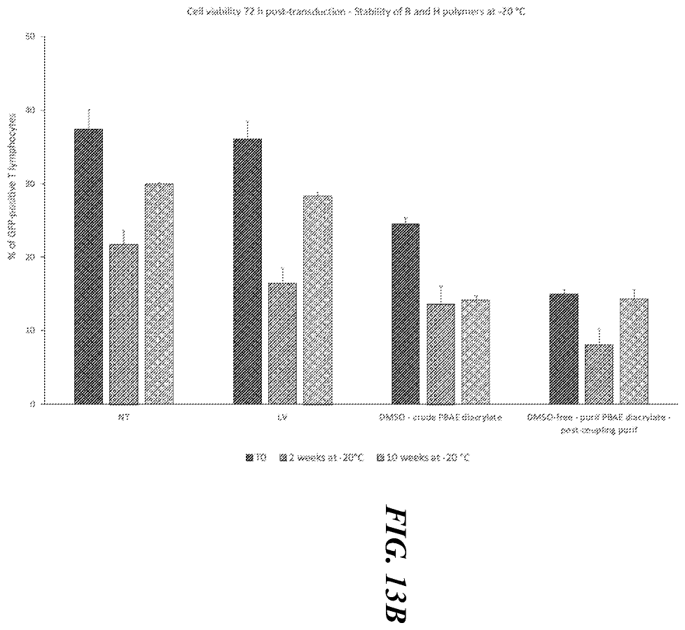

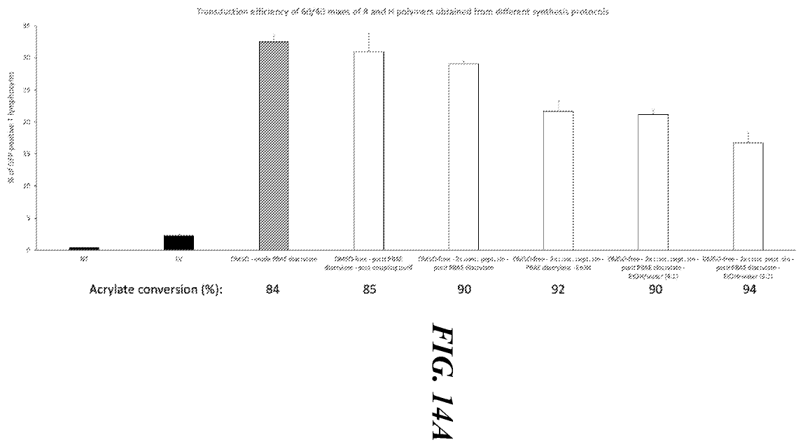

Based on these encouraging results, we evaluated the stability of 60/40 ratios of PBAE-CR3 and PBAE-CH3 obtained with DMSO-free coupling plus PBAE-diacrylate purification plus post-coupling purification (Entries 17 and 23) freshly resuspended in water or stored in water for 2 or 10 weeks at −20° C. and compared it with polymers synthetized with the classical protocol (Entries 2 and 3) and stored in DMSO at −20° C. At the different tested time points no difference was observed in transduction efficiencies or cell toxicities between polymers obtained with the classical method and the newly implemented synthesis (see FIGS. 13A and 13B.), therefore confirming the stability of the DMSO-free polymers when stored in water at −20° C.

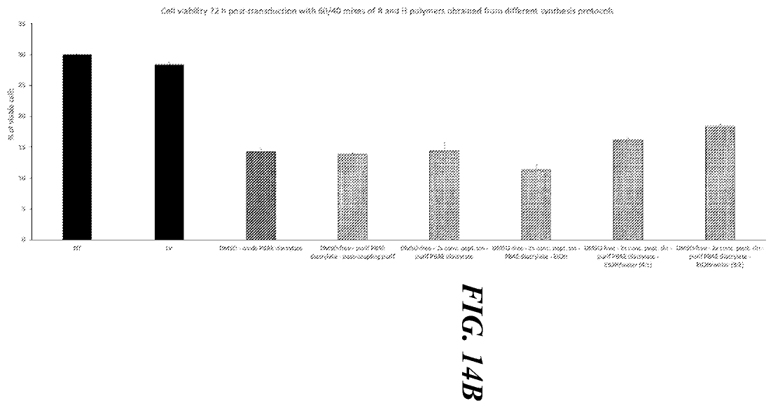

Finally, the influence of residual free acrylate content on the biological properties of PBAE-CR3, which proved to be the most challenging OM-PBAE, was evaluated in terms of peptide end-coupling efficiency. The optimization of the peptide end-coupling reaction resulted in acrylate conversion yields superior to 90% and polymers that showed transduction efficiencies or cell toxicities comparable to those obtained with the classical method. Further improvements of post-coupling purification to remove uncoupled PBAE-diacrylates translated into less toxic PBAE-CR3 derivatives but that appeared to be less interesting as transduction agents (see FIG. 14A and FIG. 14B).

Altogether, these results demonstrate that functional OM-PBAEs can be synthetized in DMSO-free conditions with less impurities and stably stored in conditions that are compatible with biological systems to provide new agents for gene delivery that are not toxic to human cells.

### REFERENCES

Anderson D J, Peng W, Akinc A, Hossain N, Kohn A Padera R et al., A polymer library approach to suicide gene therapy for cancer, Proc. Natl. Acad. Sci. USA 2004, 101:16028-33.
Anderson D G, Akinc A, Hossain N. and Langer, R. Structure/property studies of polymeric gene delivery using a library of poly(beta-amino esters). Mol. Ther. 2005, 11, 426.
de Abreu Costa L, Henrique Fernandes Ottoni M, Dos Santos M G, Meireles A B, Gomes de Almeida V et al. Dimethyl Sulfoxide (DMSO) Decreases Cell Proliferation and TNF-α, IFN-γ, and IL-2 Cytokines Production in Cultures of Peripheral Blood Lymphocytes Molecules. 2017 Nov. 10; 22(11)
Dosta P, Segovia N, Cascante A, Ramos V, Borrós S. Surface charge tunability as a powerful strategy to control electrostatic interaction for high efficiency silencing, using tailored oligopeptide-modified poly(beta-amino ester)s (PBAEs), Acta Biomaterialia. 2015 July; 20:82-93.
Galvao J, Davis B, Tilley M, Normando E, Duchen M R et al. Unexpected low-dose toxicity of the universal solvent DMSO. FASEB J. 2014, 28:1317-30
Green J J, Zhou B Y, Mitalipova M M, Beard C, Langer R et al., Nanoparticles for Gene Transfer to Human Embryonic Stem Cell Colonies. Nano letters. 2008, 8:3126-3130
Lynn D M and Langer R. Degradable Poly(β-amino esters): Synthesis, Characterization, and Self-Assembly with Plasmid DNA. JACS 2000, 122: 10761-68.
Lv H, Zhang S, Wang B, Cui S, Yan J. Toxicity of cationic lipids and cationic polymers in gene delivery. J Control Release. 2006, 114:100-9
Mangraviti A, Tzeng S Y, Kozielski K L, Wang Y, Jin Y, Gullotti D, et al. Polymeric Nanoparticles for Nonviral Gene Therapy Extend Brain Tumor Survival in Vivo, ACS Nano 2015, 9:1236-1249.
Verheijen M, Lienhard M, Schrooders Y, Clayton O, Nudischer R et al. DMSO induces drastic changes in human cellular processes and epigenetic landscape in vitro. Scientific reports. 2019, 9:4641.
Zugates, G T, Tedford N C, Zumbuehl A, Jhunjhunwala S, Kang C S, et al. Gene delivery properties of end-modified poly(beta-amino ester)s. Bioconjugate Chem. 2007, 18:1887-96

1. A method for synthesizing an end modified polymer, the method comprising:
(a) providing an end modifier and a polymer comprising a terminal acrylate group comprising a terminal vinyl carbon;
(b) forming a first solution comprising the polymer dissolved in acetonitrile;
(c) forming a second solution comprising the end modifier dissolved in an aqueous citrate solution; and
(d) mixing the first and second solutions, whereby the end modifier bonds to the terminal vinyl carbon to form the end modified polymer.

2. The method of claim 1, wherein the second solution further comprises acetonitrile.

3. The method of any of claim 2, wherein the second solution is formed by mixing the end modifier with an aqueous citrate solution until the end modifier is dissolved in the solution, then adding acetonitrile to the solution.

4. The method of claim 2 or claim 3, wherein the second solution comprises water/acetonitrile in a ratio from about 1/1 volume/volume to about 2/1 volume/volume.

5. The method of any of the preceding claims, wherein the second solution comprises about 25 mM citrate and has a pH of about pH 5.0.

6. The method of any of the preceding claims, wherein the mixing of the first and second solutions in step (d) forms a solution comprising acetonitrile/water at a ratio of about 3/2 volume/volume.

7. The method of any of the preceding claims, wherein the end modifier comprises a thiol and the end modifier bonds to the terminal vinyl carbon through a thioether bond (—C—S—C—).

8. The method of any of the preceding claims, wherein the end modifier is an oligopeptide selected from the group consisting of CRRR (SEQ ID NO:4), CKKK (SEQ ID NO:7), CHHH (SEQ ID NO:1), CDDD (SEQ ID NO:13), CEEE (SEQ ID NO:10), GRKKRRQRRRPQ (TAT) (SEQ ID NO:48), RQIKIWFQNRRMKWKKGG (penetratin) (SEQ ID NO:49), CGYGPKKKRKVGG (NLS sequence) (SEQ ID NO:50), AGYLLGKINLKALAALAKKIL (transportan10) (SEQ ID NO:51), RGD, KETWWETWWTEWSQPKKKRRV (pep-1) (SEQ ID NO:52), KLALKLALKALKAALKLA (MAP) (SEQ ID NO:53), RRRRNRTRRNRRRVR (FHV coat) (SEQ ID NO:54), and LLIILRRRIRKQAHAHSK (pVEC) (SEQ ID NO:55).

9. The method of any of the preceding claims, wherein the end modifier is selected from the group consisting of cysteine, homocysteine, and oligopeptides comprising cysteine or homocysteine, wherein the oligopeptide contains not more than 20 amino acids.

10. The method of claim 8 or claim 9, wherein the mixing of the first and second solutions forms a solution comprising thiol/acrylate at a molar ratio in the range from about 2.2/1 to about 3/1.

11. The method of claim 8 or claim 9, wherein the oligopeptide is provided as a hydrochloride salt, an acetate salt, a TFA salt, a formate salt, or a combination thereof.

12. The method of any of the preceding claims, wherein both the first solution and the second solution do not contain DMSO.

13. The method of any of the preceding claims, wherein the mixing of the first and second solutions in step (d) is carried out in an inert atmosphere, wherein the inert atmosphere reduces formation of di-sulfide during the mixing.

14. The method of any of the preceding claims, wherein the mixing of the first and second solutions in step (d) is carried out for about 20 hours.

15. The method of any of the preceding claims, wherein the mixing the first and second solutions in step (d) is carried out at about 25° C.

16. The method of any of the preceding claims, wherein (i) the polymer comprising a terminal acrylate group is a poly (beta-amino ester) (PBAE) acrylate having a structure of Formula VI or Formula VII: 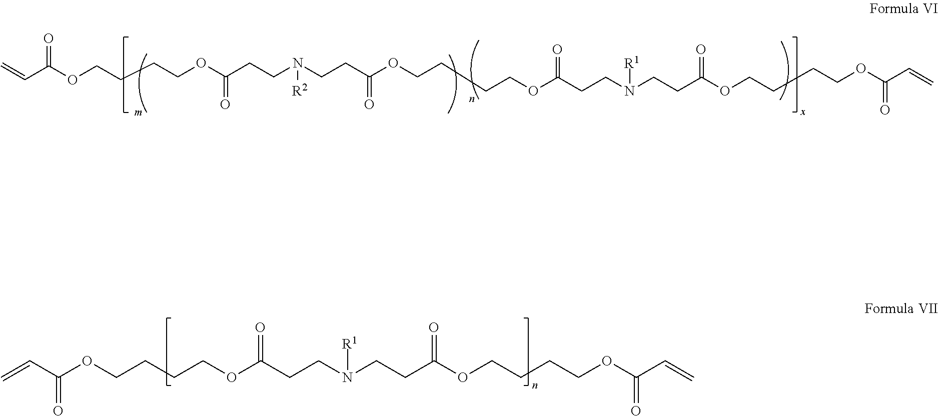
or wherein (ii) the polymer comprising a terminal acrylate group has a structure of Formula VIII, Formula IX, or Formula X: 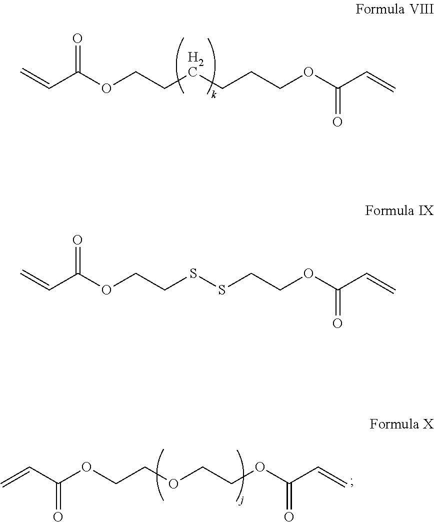
wherein R¹ and R² are each independently selected from the first group consisting of hydrogen, halogen, alkyl, alkenyl, alkynyl, phenyl, cycloalkyl, cycloalkenyl, cycloalkynyl, aryl, and heteroaryl; wherein for R¹ and R² each independently one or more carbons may be substituted by O, N, B, or S; wherein independently each constituent of the first group can optionally be further substituted with one or more substituents selected from the second group consisting of —OH, halogen, acyl halide, carbonate, ketone, aldehyde, ester, methoxy, ether, amide, amine, nitrile, and any other constituent of the first group; wherein R¹ and R² each independently have at most 20 total carbons;
wherein n and m independently are integers from 2 to 10000; k is an integer from 1 to 50000; j is an integer from 1 to 20000; and wherein X is an integer from 1 to 5000.

17. The method of any of the preceding claims, further comprising:
(e) removing residual solvents from the end modified PBAE obtained in step (d).

18. The method of claim 17, wherein the resulting end modified polymer obtained in step (e) comprises residual citrate.

19. A composition comprising an end modified polymer made by the method of any one of the preceding claims.

20. The composition of claim 19, wherein the composition is essentially free of DMSO.

21. The composition of claim 19 or claim 20 which is an aqueous solution, wherein the end modified polymer has a half-life of at least 10 weeks when the composition is stored at about −20° C.

22. The composition of claim 21, wherein the end modified polymer has a half-life of at least 15 weeks.

23. A method of purifying a polymer diacrylate, the method comprising:
(a) dissolving the polymer diacrylate in ethyl acetate;
(b) precipitating the polymer diacrylate by adding dropwise into heptane to yield a ratio of heptane to ethyl acetate of about 10/1 volume/volume; and
(c) repeating steps (a) and (b) twice, whereby the purified polymer diacrylate is obtained.

24. A method of purifying a polymer diacrylate, the method comprising:
(a) dissolving the polymer diacrylate in ethyl acetate; and
(b) precipitating the polymer diacrylate from the solution obtained in step (a) by adding heptane to the solution to yield a ratio of heptane to ethyl acetate of about 2/1 volume/volume, whereby the purified polymer diacrylate is obtained as the precipitate.

25. The method of claim 24, further comprising performing the method of claim 25 using the product of the method of claim 24 as the starting polymer diacrylate of claim 25.

26. A purified polymer diacrylate obtained by the method of any of claims 23-25.

27. A method of purifying an oligopeptide-modified PBAE (OM-PBAE), the method comprising:
(a) extracting the OM-PBAE with ethanol, and then drying the extracted OM-PBAE;
(b) re-dissolving the OM-PBAE resulting from step (a) in ethanol, and precipitating the OM-PBAE in diethylether/acetone at a ratio of about 7/3 (v/v);
(c) washing the precipitate resulting from step (b) with diethylether/acetone (about 7/3 v/v); and
(d) removing residual solvents from the OM-PBAE resulting from step (c).

28. A method of purifying an oligopeptide-modified PBAE (OM-PBAE), the method comprising:
(a) passing the OM-PBAE through a size exclusion column using an eluent comprising water;
(b) collecting the OM-PBAE after passing through the size exclusion column; and
(c) removing residual solvents from the OM-PBAE resulting from step (b).

29. The method of claim 27 or claim 28, wherein step (c) comprises applying vacuum or performing lyophilization, precipitation, filtration, centrifugation, washing the OM-PBAE with diethylether/acetone, or a combination thereof.

30. An OM-PBAE obtained by the method of any of claim 19-22 or 27-29.

31. A nanoparticle comprising a nucleic acid or a viral vector encapsulated with the OM-PBAE of claim 30.

32. The nanoparticle of claim 31, wherein the viral vector is a lentiviral vector.

33. The nanoparticle of claim 32, wherein the nanoparticle has a higher transduction efficiency compared to a nanoparticle comprising an OM-PBAE made by a method comprising the use of DMSO as solvent.

34. The nanoparticle of claim 32, wherein the nanoparticle is capable of transducing cells yielding a higher cell viability compared to a nanoparticle comprising an OM-PBAE made by a method comprising the use of DMSO as solvent.
